SEQUENCE LISTING (55 sequences)

SEQ ID NO: 1 - DNA, 15 bp, Artificial Sequence
  Description of Artificial Sequence Synthetic oligonucleotide
  modified_base (4)..(4): a, c, t, or g
  modified_base (8)..(8): a, c, t, or g
  modified_base (12)..(12): a, c, t, or g
gggngggngggnggg

SEQ ID NO: 2 - DNA, 18 bp, Artificial Sequence
  Description of Artificial Sequence Synthetic oligonucleotide
  modified_base (1)..(1): a, c, t, or g
  modified_base (5)..(5): a, c, t, or g
  modified_base (9)..(10): a, c, t, or g
  modified_base (14)..(14): a, c, t, or g
  modified_base (18)..(18): a, c, t, or g
ngggngggnngggngggn

SEQ ID NO: 3 - DNA, 20 bp, Artificial Sequence
  Description of Artificial Sequence Synthetic oligonucleotide
  modified_base (1)..(2): a, c, t, or g
  modified_base (19)..(20): a, c, t, or g
nngggtggggagggtgggnn

SEQ ID NO: 4 - DNA, 22 bp, Artificial Sequence
  Description of Artificial Sequence Synthetic oligonucleotide
tgagggtgggtagggtgggtaa

SEQ ID NO: 5 - DNA, 16 bp, Artificial Sequence
  Description of Artificial Sequence Synthetic oligonucleotide
  modified_base (4)..(4): a, c, t, or g
  modified_base (8)..(9): a, c, t, or g
  modified_base (13)..(13): a, c, t, or g
gggngggnngggnggg

SEQ ID NO: 6 - DNA, 22 bp, Artificial Sequence
  Description of Artificial Sequence Synthetic oligonucleotide
tgagggtggggagggtggggaa

SEQ ID NO: 7 - DNA, 18 bp, Artificial Sequence
  Description of Artificial Sequence Synthetic oligonucleotide
agggtggggagggtgggg

SEQ ID NO: 8 - DNA, 18 bp, Artificial Sequence
  Description of Artificial Sequence Synthetic oligonucleotide
agggtgaaaagggtgggg

SEQ ID NO: 9 - DNA, 26 bp, Artificial Sequence
  Description of Artificial Sequence Synthetic oligonucleotide
ttggggagggtggggagggtggggaa

SEQ ID NO: 10 - DNA, 27 bp, Artificial Sequence
  Description of Artificial Sequence Synthetic oligonucleotide
tggggagggtggggagggtggggaagg

SEQ ID NO: 11 - DNA, 27 bp, Artificial Sequence
  Description of Artificial Sequence Synthetic oligonucleotide
tggggagggtggaaagggtggggaagg

SEQ ID NO: 12 - DNA, 35 bp, Artificial Sequence
  Description of Artificial Sequence Synthetic oligonucleotide
agggcggtgtgggaagagggaagagggggaggcag

SEQ ID NO: 13 - DNA, 22 bp, Artificial Sequence
  Description of Artificial Sequence Synthetic oligonucleotide
agggcggtgtgggaatagggaa

SEQ ID NO: 14 - DNA, 18 bp, Artificial Sequence
  Description of Artificial Sequence Synthetic oligonucleotide
cggggggttttgggcggc

SEQ ID NO: 15 - DNA, 22 bp, Artificial Sequence
  Description of Artificial Sequence Synthetic oligonucleotide
cggggcgggccgggggcggggt

SEQ ID NO: 16 - DNA, 22 bp, Artificial Sequence
  Description of Artificial Sequence Synthetic oligonucleotide
agggagggcgctgggaggaggg

SEQ ID NO: 17 - DNA, 30 bp, Artificial Sequence
  Description of Artificial Sequence Synthetic oligonucleotide
aggggcgggcgcgggaggaagggggcggga

SEQ ID NO: 18 - DNA, 28 bp, Artificial Sequence
  Description of Artificial Sequence Synthetic oligonucleotide
cgggcgggagcgcggcgggcgggcgggc

SEQ ID NO: 19 - DNA, 19 bp, Artificial Sequence
  Description of Artificial Sequence Synthetic oligonucleotide
gggagggagagggggcggg

SEQ ID NO: 20 - DNA, 42 bp, Artificial Sequence
  Description of Artificial Sequence Synthetic oligonucleotide
ggaggaggaggtcacggaggaggaggagaaggaggaggagga

SEQ ID NO: 21 - DNA, 26 bp, Artificial Sequence
  Description of Artificial Sequence Synthetic oligonucleotide
ttagggttagggttagggttagggtt

SEQ ID NO: 22 - DNA, 26 bp, Artificial Sequence
  Description of Artificial Sequence Synthetic oligonucleotide
ttagggttagggttagggttagggaa

SEQ ID NO: 23 - DNA, 26 bp, Artificial Sequence
  Description of Artificial Sequence Synthetic oligonucleotide
gggtaggggcggggcggggcgggggc

SEQ ID NO: 24 - DNA, 48 bp, Artificial Sequence
  Description of Artificial Sequence Synthetic oligonucleotide
ggaggcgggggggggggggcgggggcgggggcgggggaggggcgcggc

SEQ ID NO: 25 - DNA, 23 bp, Artificial Sequence
  Description of Artificial Sequence Synthetic oligonucleotide
aagggggggcggcggggcaggga

SEQ ID NO: 26 - DNA, 23 bp, Artificial Sequence
  Description of Artificial Sequence Synthetic oligonucleotide
cggcggggcagggagggtggacg

SEQ ID NO: 27 - protein, 710 aa, Homo sapiens
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 28 - protein, 439 aa, Homo sapiens
PVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 29 - protein, 376 aa, Homo sapiens
PVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKP

SEQ ID NO: 30 - protein, 177 aa, Homo sapiens
MSSNECFKCGRSGHWARECPTGGGRGRGMRSRGRGGFTSDRGFQFVSSSLPDICYRCGESGHLAKDCDLQEDACYNCGRGGHIAKDCKEPKREREQCCYNCGKPGHLARDCDHADEQKCYSCGEFGHIQKDCTKVKCYRCGETGHVAINCSKTSEVNCYRCGESGHLARECTIEATA

SEQ ID NO: 31 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 32 - protein, 1249 aa, Homo sapiens
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEVTSLKYSTPPYLLEAASHLSPENFVEDEAKICVQELQCPKIITKNSPLPSSIISRKEKNDPVFLEEAGKAEKIVISRSTSPTFNKQTKRVSWSSFNSLGQYFTGKIPKATPELGSSENSASSPPRFKTEKMESKTVLPFTDKCESSNLTVNTSFGSCPQSETIISSLKIDATLTRKNHSEHPLCSEEALDPDIELSLVSEEDKQSTSNRDFETEAEDESIYFTPELYDPEDTDEEKNDLAETDRGNRLANNSDCILAKDLFEIRTIKEVDSAREVKAEDCIDTKLNGILHIEESKIDDIDGNVKTTWINELELGKTHEIEIKNFKPSPSKNKGMFPGFK

SEQ ID NO: 33 - protein, 432 aa, Homo sapiens
GGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVC

SEQ ID NO: 34 - protein, 641 aa, Homo sapiens
MLSGIEAAAGEYEDSELRCRVAVEELSPGGQPRRRQALRTAELSLGRNERRELMLRLQAPGPAGRPRCFPLRAARLFTRFAEAGRSTLRLPAHDTPGAGAVQLLLSDCPPDRLRRFLRTLRLKLAAAPGPGPASARAQLLGPRPRDFVTISPVQPEERRLRAATRVPDTTLVKRPVEPQAGAEPSTEAPRWPLPVKRLSLPSTKPQLSEEQAAVLRAVLKGQSIFFTGSAGTGKSYLLKRILGSLPPTGTVATASTGVAACHIGGTTLHAFAGIGSGQAPLAQCVALAQRPGVRQGWLNCQRLVIDEISMVEADLFDKLEAVARAVRQQNKPFGGIQLIICGDFLQLPPVTKGSQPPRFCFQSKSWKRCVPVTLELTKVWRQADQTFISLLQAVRLGRCSDEVTRQLQATASHKVGRDGIVATRLCTHQDDVALTNERRLQELPGKVHRFEAMDSNPELASTLDAQCPVSQLLQLKLGAQVMLVKNLSVSRGLVNGARGVVVGFEAEGRGLPQVRFLCGVTEVIHADRWTVQATGGQLLSRQQLPLQLAWAMSIHKSQGMTLDCVEISLGRVFASGQAYVALSRARSLQGLRVLDFDPMAVRCDPRVLHFYATLRRGRSLSLESPDDDEAASDQENMDPIL

SEQ ID NO: 35 - protein, 1417 aa, Homo sapiens
MAAVPQNNLQEQLERHSARTLNNKLSLSKPKFSGFTFKKKTSSDNNVSVTNVSVAKTPVLRNKDVNVTEDFSFSEPLPNTTNQQRVKDFFKNAPAGQETQRGGSKSLLPDFLQTPKEVVCTTQNTPTVKKSRDTALKKLEFSSSPDSLSTINDWDDMDDFDTSETSKSFVTPPQSHFVRVSTAQKSKKGKRNFFKAQLYTTNTVKTDLPPPSSESEQIDLTEEQKDDSEWLSSDVICIDDGPIAEVHINEDAQESDSLKTHLEDERDNSEKKKNLEEAELHSTEKVPCIEFDDDDYDTDFVPPSPEEIISASSSSSKCLSTLKDLDTSDRKEDVLSTSKDLLSKPEKMSMQELNPETSTDCDARQISLQQQLIHVMEHICKLIDTIPDDKLKLLDCGNELLQQRNIRRKLLTEVDFNKSDASLLGSLWRYRPDSLDGPMEGDSCPTGNSMKELNFSHLPSNSVSPGDCLLTTTLGKTGFSATRKNLFERPLFNTHLQKSFVSSNWAETPRLGKKNESSYFPGNVLTSTAVKDQNKHTASINDLERETQPSYDIDNFDIDDFDDDDDWEDIMHNLAASKSSTAAYQPIKEGRPIKSVSERLSSAKTDCLPVSSTAQNINFSESIQNYTDKSAQNLASRNLKHERFQSLSFPHTKEMMKIFHKKFGLHNFRTNQLEAINAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLYSMVHYCENITECRRIQLLAYFGENGFNPDFCKKHPDVSCDNCCKTKDYKTRDVTDDVKSIVRFVQEHSSSQGMRNIKHVGPSGRFTMNMLVDIFLGSKSAKIQSGIFGKGSAYSRHNAERLFKKLILDKILDEDLYINANDQAIAYVMLGNKAQTVLNGNLKVDFMETENSSSVKKQKALVAKVSQREEMVKKCLGELTEVCKSLGKVFGVHYFNIFNTVTLKKLAESLSSDPEVLLQIDGVTEDKLEKYGAEVISVLQKYSEWTSPAEDSSPGISLSSSRGPGRSAAEELDEEIPVSSHYFASKTRNERKRKKMPASQRSKRRKTASSGSKAKGGSATCRKISSKTKSSSIIGSSSASHTSQATSGANSKLGIMAPPKPINRPFLKPSYAFS

SEQ ID NO: 36 - protein, 349 aa, Homo sapiens
INAALLGEDCFILMPTGGGKSLCYQLPACVSPGVTVVISPLRSLIVDQVQKLTSLDIPATYLTGDKTDSEATNIYLQLSKKDPIIKLLYVTPEKICASNRLISTLENLYERKLLARFVIDEAHCVSQWGHDFRQDYKRMNMLRQKFPSVPVMALTATANPRVQKDILTQLKILRPQVFSMSFNRHNLKYYVLPKKPKKVAFDCLEWIRKHHPYDSGIIYCLSRRECDTMADTLQRDGLAALAYHAGLSDSARDEVQQKWINQDGCQVICATIAFGMGIDKPDVRFVIHASLPKSVEGYYQESGRAGRDGEISHCLLFYTYHDVTRLKRLIMMEKDGNHHTRETHFNNLY

SEQ ID NO: 37 - protein, 1008 aa, Homo sapiens
MSYDYHQNWGRDGGPRSSGGGYGGGPAGGHGGNRGSGGGGGGGGGGRGGRGRHPGHLKGREIGMWYAKKQGQKNKEAERQERAVVHMDERREEQIVQLLNSVQAKNDKESEAQISWFAPEDHGYGTEVSTKNTPCSENKLDIQEKKLINQEKKMFRIRNRSYIDRDSEYLLQENEPDGTLDQKLLEDLQKKKNDLRYIEMQHFREKLPSYGMQKELVNLIDNHQVTVISGETGCGKTTQVTQFILDNYIERGKGSACRIVCTQPRRISAISVAERVAAERAESCGSGNSTGYQIRLQSRLPRKQGSILYCTTGIILQWLQSDPYLSSVSHIVLDEIHERNLQSDVLMTVVKDLLNFRSDLKVILMSATLNAEKFSEYFGNCPMIHIPGFTFPVVEYLLEDVIEKIRYVPEQKEHRSQFKRGFMQGHVNRQEKEEKEAIYKERWPDYVRELRRRYSASTVDVIEMMEDDKVDLNLIVALIRYIVLEEEDGAILVFLPGWDNISTLHDLLMSQVMFKSDKFLIIPLHSLMPTVNQTQVFKRTPPGVRKIVIATNIAETSITIDDVVYVIDGGKIKETHFDTQNNISTMSAEWVSKANAKQRKGRAGRVQPGHCYHLYNGLRASLLDDYQLPEILRTPLEELCLQIKILRLGGIAYFLSRLMDPPSNEAVLLSIRHLMELNALDKQEELTPLGVHLARLPVEPHIGKMILFGALFCCLDPVLTIAASLSFKDPFVIPLGKEKIADARRKELAKDTRSDHLTVVNAFEGWEEARRRGFRYEKDYCWEYFLSSNTLQMLHNMKGQFAEHLLGAGFVSSRNPKDPESNINSDNEKIIKAVICAGLYPKVAKIRLNLGKKRKMVKVYTKTDGLVAVHPKSVNVEQTDFHYNWLIYHLKMRTSSIYLYDCTEVSPYCLLFFGGDISIQKDNDQETIAVDEWIVFQSPARIAHLVKELRKELDILLQEKIESPHPVDWNDTKSRDCAVLSAIIDLIKTQEKATPRNFPPRFQDGYYS

SEQ ID NO: 38 - protein, 157 aa, Homo sapiens
MSYDYHQNWGRDGGPRSSGGGYGGGPAGGHGGNRGSGGGGGGGGGGRGGRGRHPGHLKGREIGMWYAKKQGQKNKEAERQERAVVHMDERREEQIVQLLNSVQAKNDKESEAQISWFAPEDHGYGTEVSTKNTPCSENKLDIQEKKLINQEKKMFRI

SEQ ID NO: 39 - protein, 1432 aa, Homo sapiens
MSEKKLETTAQQRKCPEWMNVQNKRCAVEERKACVRKSVFEDDLPFLEFTGSIVYSYDASDCSFLSEDISMSLSDGDVVGFDMEWPPLYNRGKLGKVALIQLCVSESKCYLFHVSSMSVFPQGLKMLLENKAVKKAGVGIEGDQWKLLRDFDIKLKNFVELTDVANKKLKCTETWSLNSLVKHLLGKQLLKDKSIRCSNWSKFPLTEDQKLYAATDAYAGFIIYRNLEILDDTVQRFAINKEEEILLSDMNKQLTSISEEVMDLAKHLPHAFSKLENPRRVSILLKDISENLYSLRRMIIGSTNIETELRPSNNLNLLSFEDSTTGGVQQKQIREHEVLIHVEDETWDPTLDHLAKHDGEDVLGNKVERKEDGFEDGVEDNKLKENMERACLMSLDITEHELQILEQQSQEEYLSDIAYKSTEHLSPNDNENDTSYVIESDEDLEMEMLKHLSPNDNENDTSYVIESDEDLEMEMLKSLENLNSGTVEPTHSKCLKMERNLGLPTKEEEEDDENEANEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKLLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSTKPQEEQKTSLVAKNKICTLSQSMAITYSLFQEKKMPLKSIAESRILPLMTIGMHLSQAVKAGCPLDLERAGLTPEVQKIIADVIRNPPVNSDMSKISLIRMLVPENIDTYLIHMAIEILKHGPDSGLQPSCDVNKRRCFPGSEEICSSSKRSKEEVGINTETSSAERKRRLPVWFAKGSDTSKKLMDKTKRGGLFS

SEQ ID NO: 40 - protein, 436 aa, Artificial Sequence
  Description of Artificial Sequence Synthetic polypeptide
HSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQR

SEQ ID NO: 41 - protein, 930 aa, Artificial Sequence
  Description of Artificial Sequence Synthetic polypeptide
GHPGHLKGREIGLWYAKKQGQKNKEAERQERAVVHMDERREEQIVQLLHSVQTKNDKDEEAQISWFAPEDHGYGTEAYIDRDSEYLLQENEPDATLDQQLLEDLQKKKTDLRYIEMQRFREKLPSYGMQKELVNMIDNHQVTVISGETGCGKTTQVTQFILDNYIERGKGSACRIVCTQPRRISAISVAERVAAERAESCGNGNSTGYQIRLQSRLPRKQGSILYCTTGIILQWLQSDPHLSSVSHIVLDEIHERLQSDVLMTVVKDLLSYRPDLKVVLMSATLNAEKFSEYFGNCPMIHIPGFTFPVVEYLLEDIIEKIRYVPEQKEHRSQFKKGFMQGHVNRQEKYYYEAIYKERWPGYLRELRQRYSASTVDVVEMMDDEKVDLNLIAALIRYIVLEEEDGAILVFLPGWDNISTLHDLLMSQVMFKSDKFIIIPLHSLMPTVNQTQVFKRTPPGVRKIVIATNIAETSITIDDVVYVIDGGKIKETHFDTQNNISTMSAEWVSKANKQRKGRAGRVQPGHCYHLYNSLRASLLDDYQLPEILRTPLEELCLQIKILRLGGIAHFLSRLMDPPSNEAVLLSIKHLMELNALDKQEELTPLGVHLARLPVEPHIGKMILFGALFCCLDPVLTIAASLSFKDPFVIPLGKEKVADARRKELAAATASDHLTVVNAFKGWEKAKQRGFRYEKDYCWEYFLSSNTLQMLHNMKGQFAEHLLGAGFVSSRNPQDPESNINSDNEKIIKAVICAGLYPKVAKIRLNLGKRKMVKVYTKTDGVVAIHPKSVNVEQTEFNYNWLIYHLKMRTSSIYLYDCTEVSPYCLLFFGGDISIQKDNDQETIAVDEWIIFQSPARIAHLVKELRKELDILLQEKIESPHPVDWKDTKSRDCAVLSAIIDLIKTQEKATPRNLPPRFQDGYYSPHHHHHHHH

SEQ ID NO: 42 - DNA, 40 bp, Artificial Sequence
  Description of Artificial Sequence Synthetic oligonucleotide
ttatggggagggtggggagggtggggaaggtggggaggag

SEQ ID NO: 43 - DNA, 29 bp, Artificial Sequence
  Description of Artificial Sequence Synthetic oligonucleotide
ttggggagggtggggagggtggggaaggt

SEQ ID NO: 44 - DNA, 41 bp, Artificial Sequence
  Description of Artificial Sequence Synthetic oligonucleotide
gctgggagaagggggggcggcggggcagggagggtggacgc

SEQ ID NO: 45 - DNA, 26 bp, Artificial Sequence
  Description of Artificial Sequence Synthetic oligonucleotide
ttgggagaagggggggcggcggggca

SEQ ID NO: 46 - DNA, 19 bp, Artificial Sequence
  Description of Artificial Sequence Synthetic oligonucleotide
aagggagggcggcggggca

SEQ ID NO: 47 - DNA, 27 bp, Artificial Sequence
  Description of Artificial Sequence Synthetic oligonucleotide
aagggggggcggcggggcagggagggt

SEQ ID NO: 48 - DNA, 22 bp, Artificial Sequence
  Description of Artificial Sequence Synthetic oligonucleotide
agggttagggttagggttaggg

SEQ ID NO: 49 - DNA, 27 bp, Artificial Sequence
  Description of Artificial Sequence Synthetic oligonucleotide
ttagggttagggttagggttagggaaa

SEQ ID NO: 50 - DNA, 27 bp, Artificial Sequence
  Description of Artificial Sequence Synthetic oligonucleotide
ttagggttagggttagggttagggtta

SEQ ID NO: 51 - DNA, 42 bp, Artificial Sequence
  Description of Artificial Sequence Synthetic oligonucleotide
  misc_feature (3)..(7): This region may encompass 1-5 nucleotides
  misc_feature (11)..(15): This region may encompass 1-5 nucleotides
  misc_feature (19)..(28): This region may encompass 1-5 “ga” repeating units
  misc_feature (32)..(36): This region may encompass 1-5 nucleotides
tgaaaaagggtttttggggagagagagagggtttttggggaa

SEQ ID NO: 52 - DNA, 37 bp, Artificial Sequence
  Description of Artificial Sequence Synthetic oligonucleotide
  misc_feature (3)..(7): This region may encompass 1-5 nucleotides
  misc_feature (11)..(15): This region may encompass 1-5 nucleotides
  misc_feature (19)..(23): This region may encompass 1-5 nucleotides
  misc_feature (27)..(31): This region may encompass 1-5 nucleotides
tgaaaaagggaaaaagggaaaaagggaaaaaggggaa

SEQ ID NO: 53 - DNA, 12 bp, Artificial Sequence
  Description of Artificial Sequence Synthetic oligonucleotide
tagggttagggt

SEQ ID NO: 54 - DNA, 49 bp, Artificial Sequence
  Description of Artificial Sequence Synthetic oligonucleotide
  modified_base (1)..(5): a, c, t, or g
  misc_feature (1)..(5): This region may encompass 0-5 nucleotides
  misc_feature (7)..(11): This region may encompass 2-5 nucleotides
  modified_base (12)..(16): a, c, t, or g
  misc_feature (12)..(16): This region may encompass 0-5 nucleotides
  misc_feature (18)..(22): This region may encompass 2-5 nucleotides
  modified_base (23)..(27): a, c, t, or g
  misc_feature (23)..(27): This region may encompass 0-5 nucleotides
  misc_feature (29)..(33): This region may encompass 2-5 nucleotides
  modified_base (34)..(38): a, c, t, or g
  misc_feature (34)..(38): This region may encompass 0-5 nucleotides
  misc_feature (40)..(44): This region may encompass 2-5 nucleotides
  modified_base (45)..(49): a, c, t, or g
  misc_feature (45)..(49): This region may encompass 0-5 nucleotides
nnnnnggggggnnnnnggggggnnnnnggggggnnnnnggggggnnnnn

SEQ ID NO: 55 - DNA, 44 bp, Artificial Sequence
  Description of Artificial Sequence Synthetic oligonucleotide
  Description of Combined DNA/RNA Molecule Synthetic oligonucleotide
  modified_base (1)..(4): a, c, t, u, or g
  misc_feature (1)..(4): This region may encompass 0-4 nucleotides
  misc_feature (6)..(10): This region may encompass 2-5 nucleotides
  modified_base (11)..(14): a, c, t, u, or g
  misc_feature (11)..(14): This region may encompass 0-4 nucleotides
  misc_feature (16)..(20): This region may encompass 2-5 nucleotides
  modified_base (21)..(24): a, c, t, u, or g
  misc_feature (21)..(24): This region may encompass 0-4 nucleotides
  misc_feature (26)..(30): This region may encompass 2-5 nucleotides
  modified_base (31)..(34): a, c, t, u, or g
  misc_feature (31)..(34): This region may encompass 0-4 nucleotides
  misc_feature (36)..(40): This region may encompass 2-5 nucleotides
  modified_base (41)..(44): a, c, t, u, or g
  misc_feature (41)..(44): This region may encompass 0-4 nucleotides
nnnnggggggnnnnggggggnnnnggggggnnnnggggggnnnn

(19) United States
(12) Patent Application Publication
(10) Pub. No.: US 2021/0333284 A1
(43) Pub. Date: Oct. 28, 2021

(54) METHODS AND MATERIALS FOR LARGE-SCALE ASSESSMENT OF LIGAND BINDING SELECTIVITY OF G-QUADRUPLEX RECOGNITION USING CUSTOM G4 MICROARRAYS

(71) Applicants: Purdue Research Foundation, West Lafayette, IN (US); The United States of America, as represented by the Secretary, Department of Health and Human Servic, Bethesda, MD (US)

(72) Inventors: Danzhou YANG, West Lafayette, IN (US); Guanhui WU, West Lafayette, IN (US); Charles VINSON, Bethesda, MD (US)

(21) Appl. No.: 17/241,912

(22) Filed: Apr. 27, 2021

Related U.S. Application Data
(60) Provisional application No. 63/016,385, filed on Apr. 28, 2020.

(51) Int. Cl. G01N 33/68; C12N 15/10; G01N 21/64

(52) U.S. Cl. CPC G01N 33/68; G01N 2021/6439; G01N 21/6428; C12N 15/1093

(57) ABSTRACT

Described herein are devices and processes using single-stranded DNA sequences capable of forming G-quadruplexes (G4s) to assess the binding affinity and binding selectivity of potential G4-interactive ligands.

### CROSS-REFERENCE TO RELATED APPLICATIONS

This application claims the benefit under 35 U.S.C § 119(e) of U.S. Provisional Application No. 63/016,385 filed on Apr. 28, 2020, the entirety of the disclosure of which is incorporated herein by reference.

### GOVERNMENT RIGHTS

This invention was made with government support under CA177585 and CA023168 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 19, 2021, is named 368-15395_SL.txt and is 101,371 bytes in size.

### TECHNICAL FIELD

The invention described herein relates to the use of G-quadruplex containing microarrays to provide a large-scale assessment of ligand binding selectivity and affinity for G-quadruplexes and binding selectivity and/or affinity for individual G-quadruplexes.

### BRIEF DESCRIPTION OF THE DRAWINGS

FIG. 1 (A and B) Comparison of replicate fluorescence intensities of (A) Cy5-PDS (1 μM) and (B) Cy5-BG4 (1:100 dilution) for all 15,671 ssDNA features on the Design 3 microarray. (C and D) Comparison of (C) Cy5-PDS (0.3 μM) or (D) Cy5-BG4 fluorescence intensities in the presence of potassium (K+, x-axis, G4-stabilizing) vs lithium (Li+, y-axis, not G4-stabilizing).

FIG. 2 Summary of binding activities of proteins and molecules on the G4 microarray. A heatmap summarizing normalized fluorescence intensities of Cy5-PDS, Cy5-BG4, and 11 proteins (columns) binding 15,671 different sequences from microarray Design 3 (rows). Intensities were normalized to the range 0 (no binding) to 1 (maximum binding). Rows and columns containing proteins were clustered using hierarchical clustering via the correlation distance metric. Different classes of sequences are labeled.

FIG. 3 Sequence effects on G4 binding. (A) Summary of correlations of loop length of the MYC Pu22 G4 vs binding intensity for all molecules. (B-E) Boxplot showing binding intensity vs loop length for (B) Cy5-PDS, (C) BG4, (D) NCL (N-term del), and (E) FANCJ. Horizontal dashed line shows the binding intensity of the consensus MYC Pu22 sequence. (F) Sequence logos obtained for the 10% strongest bound variants of the loop sequence variants GGGNGGGNGGGNGGG (SEQ ID NO: 1) (Variant A) and NGGGNGGGNNGGGNGGGN (SEQ ID NO: 2) (Variant B) for Cy5-PDS, IGF2, and NCL3. (G) Sequence logos obtained for the top 10% strongest bound MYC Pu22 tail variants (NNGGGTGGGGAGGGTGGGNN (SEQ ID NO: 3)) for the indicated small molecules and proteins.

FIG. 4 Plot of Cy5-PDS binding vs DC-34 specificity (Cy5-PDS/(Cy5-PDS+DC-34). Each feature is shaded by guanine content.

FIG. 5 Western blots of protein constructs shown in TABLE 3.

FIG. 6 The binding of 3,6-bis(1-Methyl-4-vinylpyridinium) carbazole diiodide (BMVC) to various G4 structures differs from pyridostatin (PDS). Competition experiments of DNA microarrays with thousands of G4 sequences showing the differential binding of BMVC to various G4s as compared to Cy5-fluorophore (λex,max=647, λem,max=665) labeled small molecule pyridostatin (Cy5-PDS), as shown by Cy5-PDS fluorescence intensity. The competition experiments were performed in the presence of 1, 3, and 10 μM BMVC. The black dashed lines represent predicted linear relationships when the binding affinities of BMVC and PDS are the same. G4-containing sequences are shown in pale spots. Non-G4 forming sequences are shown in darker spots and serve as negative controls. Each spot represents the average of two independent measurements.

FIG. 7 Schematic diagram showing the predicted linear relationships when the competitor has the same binding affinities as Cy5-PDS. The competition effects can be revealed by a dose-dependent slope reduction.

FIG. 8 The binding preference of BMVC to known G4 structures. Among the known G4 structures, BMVC prefers to bind to MYC_14/23T, 5′-TGAGGGTGGGTAGGGTGGGTAA-3′ (SEQ ID NO: 4) (highlighted by shading). Telomeric sequences are known to form nonparallel structures [42-46] and are poorly bound by Cy5-PDS (shaded). (a) The competition microarray experiments showing dose dependent inhibitory effects of BMVC on the binding of Cy5-PDS to various known G4 structures. The G4 sequences are shown in TABLE 4. n=2 to 20 independent measurements. Error bars represent mean±SD. (b) BMVC has different inhibitory effects on the binding of Cy5-PDS to the known G4 structures at the equal molar concentration (1 μM). n=2 to 20 independent measurements. Error bars represent mean±SD

FIG. 9 The binding selectivity of BMVC for the flanking sequences of MYC G4. The inhibitory effects of BMVC on the binding of Cy5-PDS to MYC G4-derived sequences with variant 5′- and 3′-flanking segments (5′-NNGGGTGGGGAGGGTGGGNN-3′ (SEQ ID NO: 3), variant 3).

FIG. 10 The inhibitory effects of BMVC on the binding of Cy5-PDS to MYC G4-derived sequences 5′-NGGGNGGGNNGGGNGGGN-3′ (SEQ ID NO: 2), variant 4, (4096 total sequences), which include all possible loop and flanking variants.

FIG. 11 Apparent dissociation constant (K_{d, app}) of BMVC binding to various G4 structures determined by BMVC fluorescence. Conditions: 20 nM BMVC, 25° C., pH 7, 100 mM K+ (100 mM Na+ for wtTel22).

FIG. 12A Imino proton regions of the 1D 1H NMR titration spectra of BMVC with MYC_1423T G4 (a) and its 3′-end modified (b) sequence. Imino protons arising from the 1:1 or 2:1 complex formation are marked with asterisks. 2:1 complex formation, when seen, was only apparent at the highest concentration of BMVC. All spectra were collected in 95 mM K+, pH=7 solution, at 25° C. FIG. 12A discloses SEQ ID NOS 4 and 56, respectively, in order of appearance.

FIG. 12B Imino proton regions of the 1D 1H NMR titration spectra of BMVC with 5′-end modified MYC_1423T G4 sequences (c and d). Imino protons arising from the 1:1 or 2:1 complex formation are marked with asterisks. 2:1 complex formation, when seen, was only apparent at the highest concentration of BMVC. All spectra were collected in 95 mM K+, pH=7 solution, at 25° C. FIG. 12B discloses SEQ ID NOS 57 and 58, respectively, in order of appearance.

FIG. 12C Imino proton regions of the 1D 1H NMR titration spectra of BMVC with MYC_1423T G4 modified at its 5′-end (e) and its 3′-end (f). Imino protons arising from the 1:1 or 2:1 complex formation are marked with asterisks. 2:1 complex formation, when seen, was only apparent at the highest concentration of BMVC. All spectra were collected in 95 mM K+, pH=7 solution, at 25° C. FIG. 12C discloses SEQ ID NOS 59 and 60, respectively, in order of appearance.

### PART A

Both the sequence and the structure of the genome govern gene expression. Transcription factors (TFs) bind to specific double-stranded DNA (dsDNA) sequences and modulate gene expression. Sequence-specific binding of TFs to dsDNA has been observed and described for thousands of proteins.¹ However, estimates suggest that 13% of the genome has the capacity to form non-B-DNA structures.² Several proteins can bind non-B-DNA such as unfolded single stranded DNAs (ssDNA)³ and folded structures such as G4s.⁴ Understanding the factors that govern both sequence and structure-dependent binding of DNA is critical to understanding fundamental biological regulatory mechanisms. To date, it has been challenging to develop techniques capable of a high-throughput examination of the sequence specificity of non-B-DNA-binding proteins.

ssDNA containing guanine-rich stretches (G-tracts) spontaneously undergoes Hoogsteen base pairing, resulting in the formation of four-stranded structures known as G-quadruplexes (G4s).^{5,6} Physiological concentrations of potassium stabilize G4s in vitro.⁶ G4-forming DNA sequences are enriched in promoter regions of oncogenes⁷ and can be conserved across species.⁸ G4 formation has been implicated in the transcriptional regulation of oncogenes such as c-MYC⁹ and BCL2¹⁰ and are potential therapeutic targets for small molecules.¹¹ Dozens of proteins⁴ and many small molecules¹² that bind G4s have been identified. Prominent examples of small molecules include pyridostatin,¹³ 5,10,15,20-tetra(N-methyl-4-pyridyl) porphyrin (TMPyP4),¹⁴ and DC-34.¹⁵ G4-binding molecules can silence the expression of G4-associated oncogenes.¹⁵ Examples of G4-binding proteins include helicases,¹⁶ nucleolin,¹⁷ IGF2,¹⁸ and CNBP.¹⁹ Despite strong evidence for G4 formation in vivo,^{20,21} progress in understanding the G4 function has been constrained by the difficulty of examining DNA-binding specificity of molecules that bind G4s.

Most TFs bind short dsDNA sequences (6-10 nucleotides)²² allowing for the comprehensive analysis of potential binding sites.¹ Universal protein-binding microarrays (PBMs)^{23,24} have been used as a high-throughput method to determine the dsDNA-binding specificity to all possible 8-mers.¹ In contrast, the simplest G4 structure is 15 nucleotides long (i.e., GGGNGGGNNGGGNGGG (SEQ ID NO: 5)), not counting the nucleotides entering and exiting the structure (the flanking G4 tails). The types of DNA sequences known to form G4s is also expanding: several noncanonical G4s have been described including those with longer loops and/or insertions in G-tracts(bulges).²⁵ There are limits to the number of sequences that can be placed on a microarray, and thus, determining DNA binding specificity of such a large sequence space is challenging. This technology can be used to examine nearly all potential mammalian G4s, but this does not include all possible potential G4-forming sequences.

One report previously used microarrays to study about 1,900 G4-forming oligonucleotides and probed binding with a fluorescently labeled small molecule.²⁶ Microarray-based platforms for measuring G4-binding specificity have several potential advantages over sequencing-based methods. The first is that they do not require a PCR amplification step. PCR amplification is difficult for stable G4 templates, as DNA polymerase can be biochemically inhibited by G4 DNA.^{2,27,28} A second advantage is sensitivity. Protein-binding microarrays can detect distinct DNA sequence preferences between molecules even with low (<2-fold) relative differences in binding affinities.²⁹ Finally, the methods are not dependent on enrichment/pulldown efficiency: they can show that a molecule does not bind to all G4s present, whereas sequencing-based methods only detect what is efficiently pulled down.

Described herein are three Agilent DNA microarray designs that together contain a total of 24,154 unique sequences used to examine the binding specificity of proteins, antibodies, and small molecules to G4s and variants. Using Cy5-conjugated pyridostatin (Cy5-PDS) and a fluorescently labeled antibody BG4 (Cy5-BG4), it is shown that G4s can form on these microarrays, and ligand binding strength can be visualized using fluorescence imaging, validating the platform as a high-throughput method to profile G4-binding specificity. These arrays may be used to identify distinct G4-binding preferences of a panel of GST-tagged proteins (CNBP, IGF2, nucleolin, and five helicases). Finally, competition experiments between Cy5-PDS and the small molecule DC-34 reveal the G4-binding specificity of DC-34, highlighting the ability of the platform to examine DNA binding specificity of unlabeled compounds.

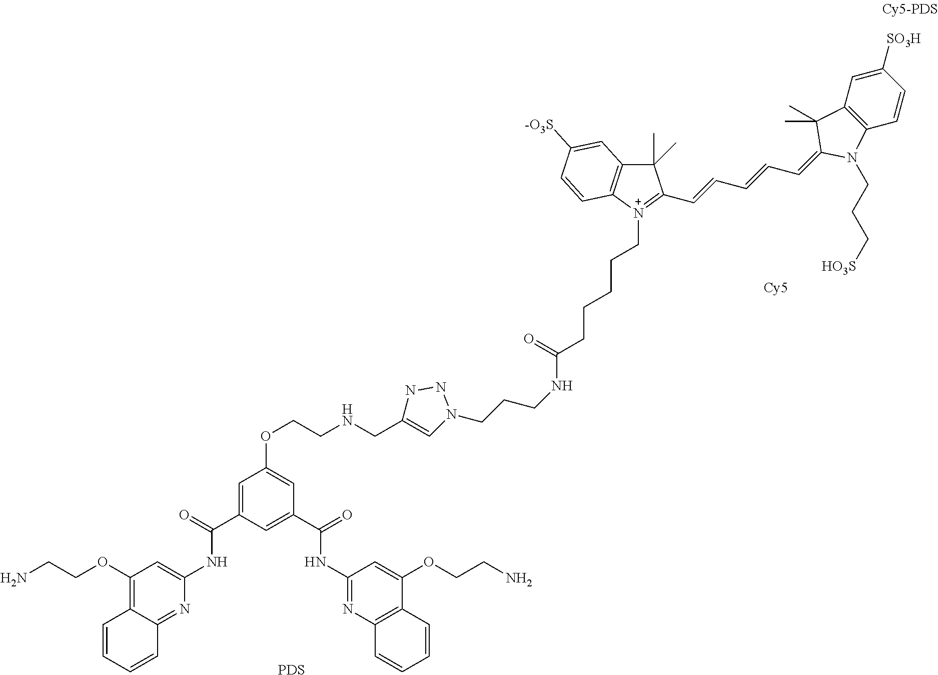

### Design of a G4 Microarray.

Three Agilent DNA microarrays (TABLE 1) were designed, each with four identical sectors that contain ca.177,440 ssDNA 60-mers to examine G4-binding specificity. Arrays were designed with 9-73 replicates of each unique sequence to ensure statistical significance (TABLE 1). Each microarray contains different sets of G4 variants designed to examine several sequence parameters that affect G4 formation and binding specificity such as loop length (Design 1), loop sequence (Design 2), tail sequence (Design 2), and single nucleotide variants of six known G4s (Design 3). All microarrays include a set of 19 sequences from human telomeres and oncogene promoters known to form G4s with various topologies as positive controls (TABLE 2). Designs 2 and 3 have a set of 295 additional G4-forming sequences from the literature.³⁰ For the loop length variants, the length of the tails and loops of four different MYC G4 sequences (MYC Pu27, MYC Pu18ntd, MYC Pu22, and MYC Pu22 NMR mutant) was increased up to five times their length. Loop and tail sequences were varied using A, T, G, and C polynucleotide stretches and a subset of combinations. For the loop sequence variants, 4,096 sequences of the form NGGGNGGGNNGGGNGGGN (SEQ ID NO: 2) and 64 variants of the form GGGNGGGNGGGNGGG (SEQ ID NO: 1) were generated. For the tail variants, 256 versions of the major MYC G4 with all possible dinucleotide tails (NNGGGTGGGGAGGGTGGGNN (SEQ ID NO: 3)) were generated. All single nucleotide variations at all positions of eight previously characterized G4 sequences (MYC Pu22, PDGFRβ, BCL2, and human telomeric G4) were generated (TABLE 1). Negative controls include 19 oncogene G4s in which all G tracts are replaced with either A, T, or C, reverse complements of G4 sequences, as well as a set of 86 published non-G4 sequences³⁰ (TABLE 1). Design 3 is the most comprehensive of the three designs, which contains sequences found in Designs 1 and 2 as well as additional G4 sequences. This design was used for most of the experiments and analyses described herein.

TABLE 1
Summary Of Array Designs

| SEQUENCE TYPE | DESIGN 1 | DESIGN 2 | DESIGN 3 |
|---|---|---|---|
| G4 variants | loop length variants | tail sequence | tail sequence |
| | of MYC G4s | NNGGGTGGGGAGGGTGGGNN | |
| | | (SEQ ID NO: 3) | |
| | G4 location (surface | loop sequence | loop length variants of MYC G4s |
| | vs buried) | | |
| | | NGGGNGGGNNGGGNGGGN | nucleotide variations of known G4 |
| | | (SEQ ID NO: 2), | (MYC, Bcl2, Telomeric, PDGFR) |
| | | GGGNGGGNGGGNGGG (SEQ | |
| | | ID NO: 1) | |
| positive | human oncogene G4s | human oncogene G4s | human oncogene G4s |
| controls | | G4 sequences from ref 30 | G4 sequences from ref 30 |
| negative | replacement of G- | replacement of G-tracts | replacement of G-tracts with (A/C/T) |
| controls | tracts with (A/C/T) | with (A/C/T) | |
| | | non-G4 sequences from ref | non-G4 sequences from ref 30 |
| | | 30 | |
| | | reverse complements of G4 | reverse complements of G4 sequences |
| | | sequences | randomly selected from Universal PBM |
| | | | (GEO platform GPL11260) |
| no. of 60 mer | 2,264 | 18,512 | 15,671 |
| sequences | | | |
| (no. of | (73 replicates) | (9 replicates) | (15 replicates) |
| replicates) | | | |

TABLE 2
Human Oncogene G4s

| | SEQ ID | | Topology if |
|---|---|---|---|
| Name | NO: | Sequence | known |
| MYC Pu22 | 6 | TGAGGGTGGGGAGGGTGGGGAA | Parallel |
| MYC 18ntd | 7 | AGGGTGGGGAGGGTGGGG | Parallel |
| MYC 18ntd mutant | 8 | AGGGTGAAAAGGGTGGGG | Parallel |
| MYC Pu26 | 9 | TTGGGGAGGGTGGGGAGGGTGGGGAA | Parallel |
| MYC Pu27 | 10 | TGGGGAGGGTGGGGAGGGTGGGGAAGG | Parallel |
| MYC Pu27 Mutant | 11 | TGGGGAGGGTGGAAAGGGTGGGGAAGG | Parallel |
| MYC Pu22 Mutant | 4 | TGAGGGTGGGTAGGGTGGGTAA | Parallel |
| NMR | | | |
| KRAS | 12 | AGGGCGGTGTGGGAAGAGGGAAGAGGGGGAGGCAG | Parallel |
| KRAS NMR | 13 | AGGGCGGTGTGGGAATAGGGAA | Parallel |
| rb1 | 14 | CGGGGGGTTTTGGGCGGC | Anti-parallel |
| VEGF | 15 | CGGGGCGGGCCGGGGGCGGGGT | Parallel |
| c-KIT | 16 | AGGGAGGGCGCTGGGAGGAGGG | Parallel |
| BCL2 Pu30/55G | 17 | AGGGGCGGGCGCGGGAGGAAGGGGGCGGGA | Parallel |
| BCL2 P1G4 | 18 | CGGGCGGGAGCGCGGCGGGCGGGCGGGC | Parallel |
| HIF1a | 19 | GGGAGGGAGAGGGGGCGGG | Parallel |
| MYB | 20 | GGAGGAGGAGGTCACGGAGGAGGAGGAGAAGGAGGAGGAGGA | Parallel |
| HuTel | 21 | TTAGGGTTAGGGTTAGGGTTAGGGTT | Hybrid/mixed |
| DNA/hTelomeric | | | |
| HuTel | 22 | TTAGGGTTAGGGTTAGGGTTAGGGAA | Hybrid/mixed |
| DNA1/hTelomeric1 | | | |
| RET | 23 | GGGTAGGGGCGGGGCGGGGCGGGGGC | Parallel |
| PDGF-A Pu48 | 24 | GGAGGCGGGGGGGGGGGGGCGGGGGCGGGGGCGGGGGAGGGGCGCGGC | Parallel |
| PGDFRβ Pu23 5′mid | 25 | AAGGGGGGGCGGCGGGGCAGGGA | Parallel |
| PGDFRβ 3′end | 26 | CGGCGGGGCAGGGAGGGTGGACG | Parallel |

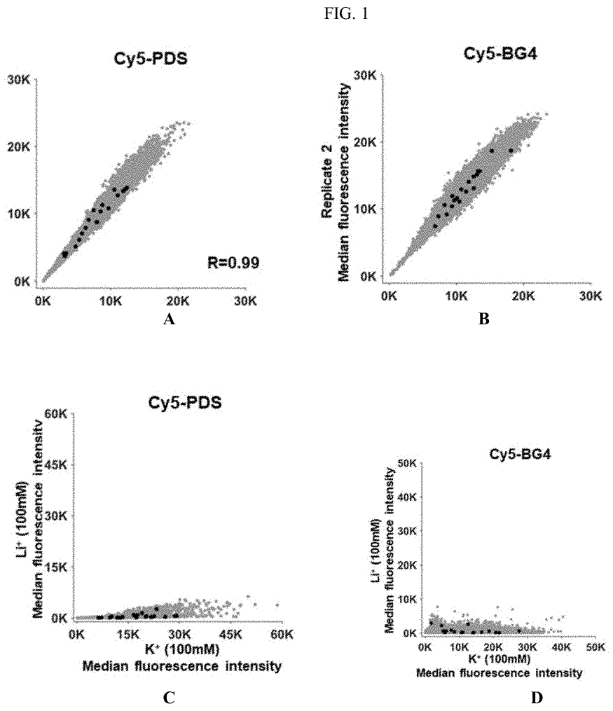

The binding specificities of several molecules were evaluated. Microarrays were preincubated with 100 mM potassium chloride to induce G4 formation. Binding of each molecule is measured by detection of fluorescence intensity at each of the microarray features. BG4 and pyridostatin were conjugated with Cy5. Cellular proteins were expressed as chimeric proteins containing GST, and binding for these proteins was detected using an anti-GST antibody conjugated with Cy5 (Materials and Methods). G4 Structures Fold on DNA Microarrays. To evaluate the utility of DNA microarrays to examine G4-binding specificity, a Cy5-labeled pyridostatin (Cy5-PDS), a small molecule known to bind broadly to G4 structures, was synthesized.¹³ A Cy5 conjugated version of BG4 (Cy5-BG4), an antibody developed to bind G4s, was also obtained.³¹ FIG. 1A, B presents replicate binding intensities to 15,491 DNA sequences on the Design 3 microarray using either Cy5-PDS or Cy5-BG4. For Cy5-PDS, robust binding is observed at 1 μM, with fluorescence intensities ranging over 100-fold between strongest and weakest bound DNA features. The fluorescence-binding intensities of Cy5-PDS are proportional to the concentration of pyridostatin used). For Cy5-PDS, strong binding was observed for 19 known genomic G4s, whereas negative controls (oligonucleotides incapable of folding into G4s) have over 100-fold lower binding, consistent with preferential Cy5-PDS binding only to G4 structures. In contrast, Cy5-BG4 binds G4-forming sequences, but it also binds several ssDNA sequences on the microarray incapable of forming G4s (FIG. 1B). Antibody binding to non-G4 features increases with higher concentrations, and in some cases non-G4 sequences are more strongly bound by Cy5-BG4 than G4 sequences, including multiple cytosine-rich negative control sequences. Inhibition of G4 Formation Inhibits Cy5-PDS and Cy5-BG4 Binding. Cy5-PDS and Cy5-BG4 binding under conditions that inhibit G4 formation was examined to evaluate if G4 structures form on the microarray and are required for binding. In one experiment, potassium chloride (which stabilizes G4s) was replaced with lithium chloride (which does not stabilize G4s)^{28,32} and a decrease in binding was observed for both Cy5-PDS (FIG. 1C) and Cy5-BG4 (FIG. 1D). Both Cy5-PDS and Cy5-BG4 showed preferred binding in a potassium solution that stabilizes G4 formation. It is noted that many sequences, in addition to the oncogene G4 sequences, are capable of forming G4s. Cy5-PDS binding to genomic G4s decreased up to 9-141-fold (>30-fold on average, FIG. 1C,) in lithium solution, while binding to negative controls decreased only 2-30-fold (FIG. 1C,), suggesting that Cy5-PDS specifically binds folded G4s rather than G-rich sequences. For Cy5-BG4, the decrease in binding was up to 270-fold for genomic G4 sequences, while binding to negative controls decreased up to 23-fold (FIG. 1D,). In a second experiment, Cy5-PDS binding following a primer extension reaction that produces dsDNA²⁴ was examined (see Materials and Methods), anticipating that dsDNA would predominate over G4 formation¹³. Formation of dsDNA for each microarray feature was quantified using a spike-in of fluorescently labeled cytosine (Cy3-dCTP).³³ Many features did not incorporate Cy3-dCTP but retained Cy5-PDS binding, suggesting that dsDNA was not produced. These features tend to be guanine-rich and contain known G4 sequences, suggesting that G4 structures form on the microarray and inhibit T7-DNA polymerase processivity, consistent with previous observations. 2,27,28

### Protein-Binding Specificity to G4 DNA in Potassium and Lithium.

The G4-binding specificity of eight GST-tagged human cellular proteins: two nucleolin (NCL) constructs (an N-terminal deletion of amino acid residues 1-271 and the RNA-recognition motifs (RRMs) only, i.e., residues 272-647), CNBP, IGF2, and full-length and truncated versions of 5 human helicases were examined. Each protein construct bound G4 microarray features in the presence of potassium. Similar binding of IVT-expressed or purified helicase DHX36 was observed. Lithium chloride weakened binding for most proteins (IGF2, NCL, FANCJ, BLM, WRN, and DHX36), highlighting their preference for binding folded G4 structures. An effect of the specific cation (potassium or lithium) on protein binding cannot be ruled out, as a reduction in binding to negative control sequences was also observed for these proteins, similar to that observed for Cy5-BG4, CNBP, and the DNA-binding domain of FANCJ, and to a lesser extent PIF1. CNBP binding to lithium treated microarrays and dsDNA is consistent with previous reports that CNBP binds guanine-rich nucleic acids.¹⁹

### Diversity of G4-Binding Specificity of Cellular Proteins.

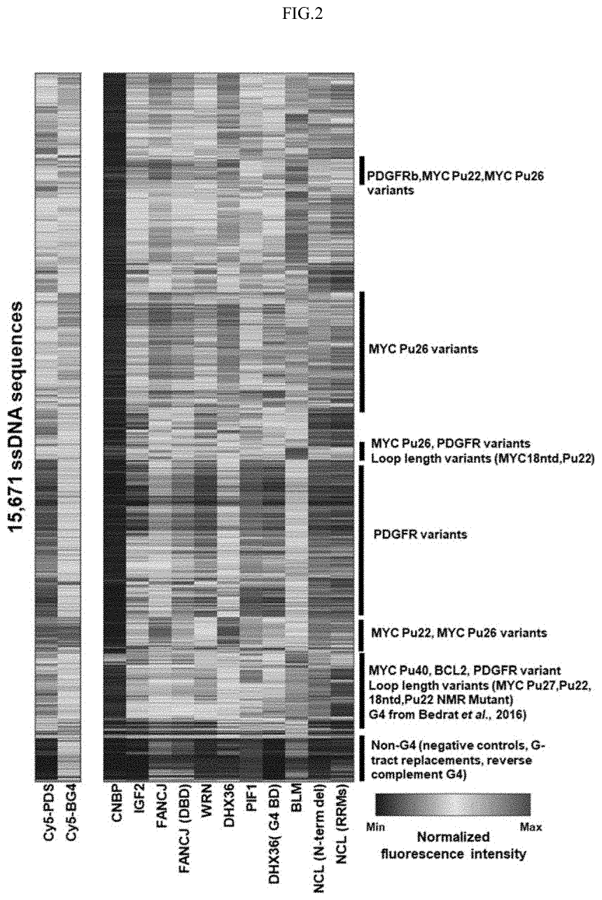

FIG. 2 presents a heatmap summarizing the different G4-binding specificities of 13 molecules to sequences on the Design 3 microarray. All molecules bind different groups of G4s. For example, Cy5-PDS preferentially binds G4 with specific sequence properties and topologies, including sequences with more than 4 G-tracts and parallel G4s (i.e., MYC Pu40, MYC Pu22, VEGF, PDGFRβ, RET, BCL2-Pu3055G, and BCL2-P1G4, TABLE 2). Moderate to low binding intensities were obtained for G4s with mixed/hybrid (hTelomeric, hTelomeric1) or antiparallel (CEB1³⁴) topologies (FIG. 2). Notably, the binding profile of Cy5-BG4 is distinct from Cy5-PDS (FIG. 2). Two distinct binding preferences in this panel of molecules were identified: those that bind only G4 sequences (i.e., IGF2 and the helicase DHX36 and those that also bind other ssDNAs in addition to folded G4s (i.e., BG4, nucleolin, and FANCJ). For example, similar to Cy5-BG4, nucleolin preferably binds folded G4 structures, as previously reported.¹⁷ However, the two proteins also appear to bind non-G4 sequences, with nucleolin to a lesser extent, as shown by both potassium-lithium preference and comparison with Cy5-PDS.

### The Effect of Single Nucleotide Variants on G4 Binding.

The utility of the microarray platform to detect how single nucleotide variants (SNVs) of known G4s affect binding was assessed. Cy5-PDS binding the MYC Pu22 G4 was examined with the expectation that variation of the nucleotides that are important to the G4 structure would result in weaker binding. In general, it was found that alteration of the guanine repeats results in weaker binding, with the largest effect occurring in the central guanine of each G-tract. In the MYC Pu22 G4, there are two G-tracts (positions 8-11 and 17-20) that are four nucleotides long. Sequences with variants at G9 and G10 are more weakly bound by Cy5-PDS, suggesting they participate in one of the four strands of the G4. In contrast, G8 and G11 can accommodate other bases, suggesting that guanine trinucleotides comprised of positions 8-10 or 9-11 can participate in the G4 structure. For the second G-tract, variants of G20 are better bound than the consensus suggesting it is not in the G4 structure, while variants of G17, G18, and G19 are more weakly bound, suggesting they are the guanine trinucleotide that is part of the G4 structure, consistent with previous reports.^{35,36} Variations in the loops (positions 7, 12, and 16) and tail sequences can either weaken or strengthen Cy5-PDS, with cytosine or thymine being preferred in the loops. Examination of Cy5-PDS binding SNVs of five other G4 sequences (MYC Pu26, BCL2 P1G4, BCL2 55G, hTelomeric, and hTelomeric1) also highlighted G-tracts participating in the G-tetrad for all sequences except for the hTelomeric sequence. For example, in the G-rich BCL2 P1G4, which contains five G-tracts, the four G-tracts participating in the G4 structure were identified and the long (12 nucleotides) second loop, consistent with previous reports.³⁷ Variations affect Cy5-PDS binding for the hTelomeric G4 sequence differently, in which nucleotide substitutions at most positions increase Cy5-PDS binding. This G4 differs from the hTelomeric1 G4 sequence only at the dinucleotide at the 3′ tail (TTAGGGTTAGGGTTAGGGTTAGGGTT (SEQ ID NO: 21) for the hTelomeric G4 versus TTAGGGTTAGGGTTAGGGTTAGGGAA (SEQ ID NO: 22) for hTelomeric1). These results are indicative of the interplay of the 3′ end and other nucleotides of the sequence in determining the G4 structure and Cy5-PDS binding, consistent with previous results suggesting that these nucleotides affect the structure of the telomeric G4.³⁸ Examination of single nucleotide variants of longer G4s such as the MYC Pu40 and PDGFRβ G4s, which contain more than four G-tracts and can thus potentially form multiple G4 structures, revealed that mutations of these G-tracts have variable effects on Cy5-PDS binding. Thus, different G4 structures may be forming in these sequences.

Several truncations of the PDGFRβ G4, which contain only four G-tracts, were examined and it was found that Cy-5 PDS can bind each truncation.

Examination of protein binding to SNVs of a panel of G4s identifies unique patterns and provides base resolution data for investigators interested in G4 structure and G4-protein interactions. For example, mutations of the G-tracts of the MYC Pu26 G4 reduce binding of all proteins examined except for in the case of PIF1, in which all variants increase binding. Another example is the effect of variations of hTelomeric and hTelomeric1 G4s on BLM binding. Here, SNVs have opposite effects on BLM binding, similar to that observed for Cy5-PDS. However, unlike Cy5-PDS, the binding pattern is reversed: substitutions of the G-tracts of the hTelomeric sequence decrease BLM binding, whereas sequence variations at most positions of the hTelomeric1 G4 increase BLM binding, suggesting that G4 topology may be an important determinant of BLM-binding specificity and function.

### Effects of G4 Loop and Tail Parameters on Molecule Binding.

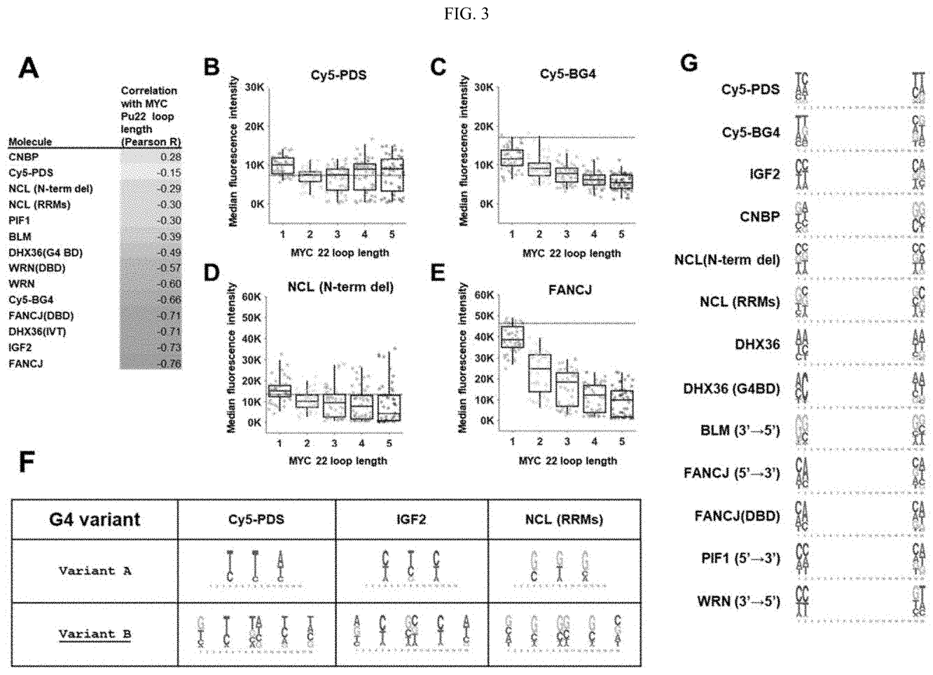

The effect of specific sequence parameters on molecule binding was examined. Loop length (Designs 1 and 3) and sequence (NGGGNGGGNNGGGNGGGN (SEQ ID NO: 2), Design 2) were examined, both of which influence G4 stability³⁹ and topology.⁴⁰ FIG. 3A summarizes the correlation of loop length of the MYC Pu22 G4 sequence on the binding of each molecule. While loop length does not appear to affect binding of Cy5-PDS (R=−0.15), binding decreases with increasing loop length for most molecules including Cy5-BG4 (R<−0.29, FIGS. 3A-E), with the strongest effect observed for the helicase FANCJ. This suggests that longer loops disrupt the protein-DNA interface. Multiple sequences with long loops that are bound better than the parental sequence by several molecules and proteins (dotted horizontal line of FIGS. 3B-E) were identified. For example, Cy5-PDS preferentially binds MYC Pu22 G4s with loops >2 nucleotides long comprised primarily of poly-G or poly-T stretches. An examination of all possible loop sequence variants of a simple G4 (GGGNGGGNGGGNGGG (SEQ ID NO: 1), 64 variants) and a MYC Pu22-like G4 sequence (NGGGNGGGNNGGGNGGGN (SEQ ID NO: 2), 4,096 variants) further highlights differences between proteins and Cy5-PDS. For example, Cy5-PDS binds both classes of sequences over a 2-3-fold range. Distinct patterns within the best-bound sequences were identified, including flexibility for the nucleotides in the central loop of the G4 and an overall preference for thymines in loops (FIG. 3F), consistent with previous findings that T nucleotides in loops have a greater propensity for folding into G4s than other nucleotides.³⁹ Different tail sequence (NNGGGTGGGGAGGGTGGGNN (SEQ ID NO: 3)) preferences were found for all measured molecules (FIG. 3G), further underscoring the utility of the platform in identifying sequence features important for G4 binding and highlighting tail sequences in determining binding specificity. For example, DHX36 preferentially binds MYC22 G4 variants containing pyrimidines (C/T) at the 5′ end of the G4, whereas a lack of sequence specificity was observed for the 3′ end. This is consistent with the published DHX36 crystal structure that highlighted the DHX-specific motif interacting with the 5′ tail and surface of the MYC Pu22 G4.⁴¹

### Competition Experiments Reveal G4-Binding Specificity of Unlabeled Small Molecules.

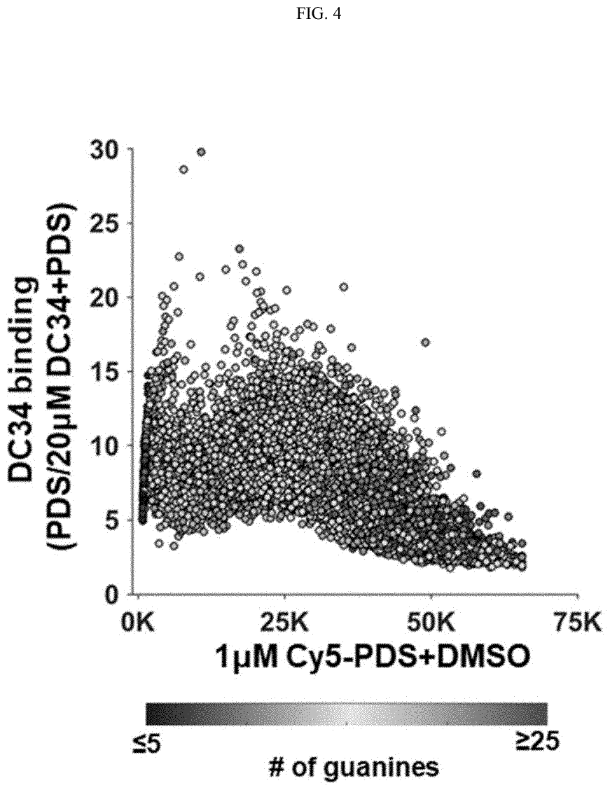

Whether the microarray platform could be used to reveal the G4-binding specificity of unlabeled molecules via a competition with Cy5-PDS binding was explored. Three example molecules, unlabeled PDS, TMPyP4 (a planar molecule that nonspecifically binds G4 structures¹⁴), and DC-34 (a molecule that selectively binds the MYC G4¹⁵) were examined. A competition experiment with unlabeled pyridostatin indicates no change in binding specificity, with weaker-bound G4s being more easily competed. Comparison of 1 μM Cy5-PDS binding in the presence or absence of various concentrations of unlabeled TMPyP4 indicated a uniform reduction in Cy5-PDS binding to all G4-containing features. These results confirm that TMPyP4 nonspecifically competes with Cy5-PDS for binding to all G4s. The binding of unlabeled DC-34 was examined (FIG. 4). There appear to be no features that are better-bound in the presence of DC-34. Instead, some features are poorly bound by Cy5-PDS in the presence of DC-34, suggestive of specific DC-34 binding. Specifically, 17.5% of G4 sequences decreased in intensity greater than 10-fold, suggesting that DC-34 competitively binds to only a subset of the G4s. Similar results were observed with higher concentrations of DC-34. The difference in Cy5-PDS binding to variants in the tails of the MYC Pu22 G4 in the presence of DC-34 is 3-fold, with sequences containing purine (A or G) directly adjacent to the G4 structure being preferentially bound by DC-34 (i.e., they have the strongest reduction in Cy5-PDS binding in the presence of DC-34). This is consistent with the observation that DC-34 binds the top and bottom surfaces of the G4 and makes specific contacts with purines in the tail sequences.¹⁵ The general properties of features in which DC-34 reduced binding of Cy5-PDS (ratio of PDS/PDS+DC-34) were also examined. DC-34 appears to preferentially bind features that are moderately bound by PDS (variants of telomeric G4s) and those that tend to have signatures of less stable G4s, such as moderate dCTP incorporation and moderate G-content.

Measurements of G4 Binding on Microarrays Correlate with Sequencing-Based Methods.

How well the microarray-based measurements for Cy5-PDS binding correlate with G4 stability measured using high-throughput sequencing was evaluated.²⁸ A method (G4Detector)⁴² that uses parameters learned from high-throughput sequencing-based measurements of hundreds of thousands of human G4 occurrences²⁸ to predict microarray intensities based on the probe sequence was applied. The predicted intensities show a high positive correlation with the measured array intensities for Cy5-PDS binding (R=0.61, p-value <1e-15), indicating good agreement between PDS-binding measurements made using either microarray or sequencing-based technologies. These results further demonstrate the generalizability of using the array-based measurements described herein: although the model used was trained on human genomic sequences, it appears to have good predictive power on unrelated sequences (i.e. the array probes described herein).

### Discussion

Use of microarrays containing thousands of different ssDNA sequences to evaluate G4 DNA-binding specificity of proteins and small molecules is described herein. Previous efforts to use G4 microarrays have focused on examining the binding of labeled small molecules to ca. 2,000 G4-forming sequences.²⁶ Herein, is described the systematic assessment of protein, small molecule, and antibody binding to more than 25,000 G4 sequences, approaching the number and sequence diversity of G4s thought to exist at a given time in the human genome.²⁰ The binding preferences of a G4 antibody as well as a variety of helicases and known endogenous G4-binding proteins are demonstrated herein. Distinct and coherent patterns/preferences of each molecule for different sequences even with low relative differences in intensities are found, highlighting the sensitivity of the approach. Also demonstrated is that in competitive assays, the selectivity of unlabeled small molecules can also be assessed, revealing a label-free method for quantifying G4-binding specificity. This work highlights the utility of the microarray platform to assess the specificity of G4-binding molecules. For example, BG4 is an antibody developed to bind G4s³¹ and has been used to examine occurrences of the G4 structure in vivo.²¹ The G4-binding specificity of BG4 has only been validated using a handful of sequences.³¹ Examination of Cy5-BG4 binding to the G4 microarrays described herein indicates the binding specificity of Cy5-BG4 is distinct from Cy5-PDS, a small molecule that also broadly binds G4s. It has been discovered that unlike Cy5-PDS, Cy5-BG4 G4 has the capacity to bind to some unfolded and non-G-tract containing ssDNA sequences, including multiple cytosine-rich sequences. Still, the possibility exists that BG4 induces a G4-like fold in some G-rich ssDNA sequences. Analysis of the effect of loop lengths on binding indicates that Cy5-BG4 preferentially bind G4s with short loops, unlike Cy5-PDS, which binds similarly to G4 sequences with various loop lengths. Because BG4 does not bind to all G4s, it is possible that pulldown assays such as ChIP-seq with BG4 may either underrepresent or overrepresent the occurrence of G4s in cells or lysates. Thus, caution should be exercised in considering pulldown assays with BG4. Experiments using this approach can also provide insights into G4-mediated regulation of biological processes. Transcription initiation is a dynamic process that involves several mechanical and topological changes to dsDNA.⁴³ It has been demonstrated that the use of microarray platforms can distinguish the binding specificity of a given molecule or protein for structured or linear DNAs. For example, examination of protein binding in the presence of lithium (disfavoring G4 formation) in comparison with potassium (stabilizing G4 formation) demonstrates that inhibiting G4 formation does not inhibit DNA binding of the known G4-binding proteins CNBP and PIF1. It may therefore be more appropriate to consider these proteins as binding to purine-rich sequences of multiple conformations. It may be that the flexibility in binding DNA in multiple conformations may allow these proteins to bind genomic regions undergoing transitions in DNA conformation. In contrast, proteins such as IGF2 and DHX36 only bind to folded G4 sequences. IGF2 traditionally is known to act extracellularly, binding to the surface of cells and activating multiple signaling pathways.⁴⁴ The possibility that it also functions by directly binding to DNA is another example of a protein having multiple functions by binding totally unrelated cellular components.⁴⁵ hTelomeric G4 is structurally polymorphic which may be important for its function. Interestingly, the data disclosed herein shows that BLM specifically binds the wt hTelomeric sequence that forms hyb-2 G4, while WRN can bind both hTelomeric (hyb-2 G4) and hTelomeric1 (hyb-1 G4) sequences, suggesting that G4 topology may be an important determinant of different binding specificities and functions of BLM and WRN. The differences in binding to G4 sequences between proteins and Cy5-PDS also suggest that they may recognize distinct surfaces of the G4 structure. Analysis of future structures of small molecules and proteins in complex with G4 DNA such as the one already described⁴¹ may aid in understanding the array data, such as the contribution of different SNVs to binding specificity.

In conclusion, it is shown that the microarray-based analysis of G4-binding events is a robust and sensitive technology to examine DNA-binding specificity of small molecules and proteins to tens of thousands of ssDNA structures including G4s in a single experiment. The data provide a rich resource for investigators interested in noncanonical nucleic acid structures and G4 molecule-binding specificity. The customizability and flexibility in using microarrays to examine various aspects of G4 structure, stability, and binding by small molecules and proteins is highlighted by this work. Many G4s are polymorphic and have topologies dependent on temperature,⁴⁶ cation identity (K+, Na+, or Li+), or concentrations.³² The results disclosed herein anticipate experiments conducted using differing conditions (salt concentrations or alternative ions) for the determination of aspects of G4 formation and stability. Parameters affecting cooperative G4-binding specificity can be examined via additional custom array designs in which the number of G-tracts within a DNA probe is varied systematically. Finally, the platforms described herein present a unique approach to understanding the sequence and structure parameters that govern nucleic acid recognition by antibodies, proteins, and small molecules in an unbiased format.

### Materials and Methods

Synthesis of Cy5 Conjugated Pyridostatin. To a 1-dram vial was added alkynyl pyridostatin (1.0 mg, 0.00102 mmol)⁴⁷ from a 5 mg mL-1 stock in DMSO. The solution was diluted with a water/tertbutyl alcohol mixture (1.0 mL, 1:1 v/v). Cy5-N3 (1.03 mg, 0.00123 mmol) was then added from a 10 mM aqueous stock solution, followed by cupric sulfate (0.065 mg, 0.00041 mmol) and sodium ascorbate (0.2 mg, 0.00102 mmol) which were added from 5 mg mL-1 aqueous stock solutions. The reaction was stirred at RT for 1 h, at which time LC/MS indicated consumption of the starting material. The reaction was diluted with water (3 mL), and the solution was directly purified by reverse-phase preparative HPLC (5-90% MeCN/0.1% aqueous (NH₄HCO₃). The product-containing fractions were lyophilized to afford Cy5-PDS (1.3 mg, 76%) as a blue solid.

### Sources of Antibody, Small Molecule, and Protein Constructs.

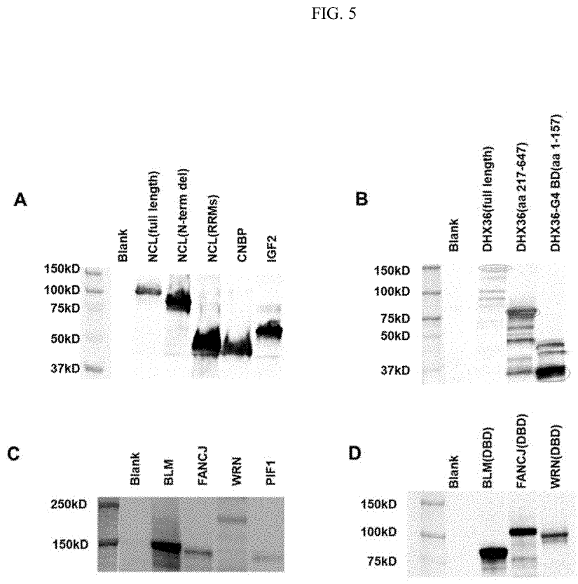

BG4,³¹ conjugated with FluoProbes647H (Cy5-BG4), was obtained from Absolute Antibody (product number Ab00174-1.1). TMPyP4 was obtained from Sigma-Aldrich (catalog number 613560). N-terminal glutathione S-transferase (GST) tagged human nucleolin IGF2, CNBP, and helicase plasmids were synthesized by GenScript. Purified, recombinant bovine DHX36⁴¹ was provided as a gracious gift by the Ferré-D'Amaré Lab (National Institutes of Health, Bethesda). The sequences of all proteins used are listed in TABLE 3. All chimeric proteins were expressed via in vitro translation (IVT) reactions using the PURExpress In Vitro Protein Synthesis Kit (NEB) as described previously.²³ For all IVT reactions, 288 ng of plasmid was added to 80 μL of a IVT mixture, and reactions were carried out at 37° C. for 2 h. Expression of all protein constructs was confirmed via Western blot (FIG. 5).

TABLE 3

| | | | | | | SEQ | Length |
|---|---|---|---|---|---|---|---|
| | | | Acces- | Descrip- | | ID | (amino |
| Name | Full name | Species | sion | tion | Amino acid sequence | NO: | acids) |
| NCL1/ | Nucleolin | Homo | NM_00538 | Full | MVKLAKAGKNQGDPKKMAPPPKEVEEDSEDEEMSEDEEDDSSG | 27 | 710 |
| NCL | (full-length) | sapiens | 1 .3 | length | EEVVIPQKKGKKAAATSAKKVVVSPTKKVAVATPAKKAAVTPGKK | | |
| | | | | ORF | AAATPAKKTVTPAKAVTTPGKKGATPGKALVATPGKKGAAIPAKG | | |
| | | | | | AKNGKNAKKEDSDEEEDDDSEEDEEDDEDEDEDEDEIEPAAMKA | | |
| | | | | | AAAAPASEDEDDEDDEDDEDDDDDEEDDSEEEAMETTPAKGKK | | |
| | | | | | AAKVVPVKAKNVAEDEDEEEDDEDEDDDDDEDDEDDDDEDDEE | | |
| | | | | | EEEEEEEEPVKEAPGKRKKEMAKQKAAPEAKKQKVEGTEPTTAF | | |
| | | | | | NLFVGNLNFNKSAPELKTGISDVFAKNDLAVVDVRIGMTRKFGYV | | |
| | | | | | DFESAEDLEKALELTGLKVFGNEIKLEKPKGKDSKKERDARTLLAK | | |
| | | | | | NLPYKVTQDELKEVFEDAAEIRLVSKDGKSKGIAYIEFKTEADAEK | | |
| | | | | | TFEEKQGTEIDGRSISLYYTGEKGQNQDYRGGKNSTWSGESKTL | | |
| | | | | | VLSNLSYSATEETLQEVFEKATFIKVPQNQNGKSKGYAFIEFASFE | | |
| | | | | | DAKEALNSCNKREIEGRAIRLELQGPRGSPNARSQPSKTLFVKGL | | |
| | | | | | SEDTTEETLKESFDGSVRARIVTDRETGSSKGFGFVDFNSEEDAK | | |
| | | | | | AAKEAMEDGEIDGNKVTLDWAKPKGEGGFGGRGGGRGGFGGR | | |
| | | | | | GGGRGGRGGFGGRGRGGFGGRGGFRGGRGGGGDHKPQGKK | | |
| | | | | | TKFE | | |
| NCL2/ | Nucleolin | Homo | NM_00538 | N-terminal | PVKEAPGKRKKEMAKQKAAPEAKKQKVEGTEPTTAFNLFVGNLN | 28 | 439 |
| NCL | N-terminal | sapiens | 1 .3 | deletion | FNKSAPELKTGISDVFAKNDLAVVDVRIGMTRKFGYVDFESAEDLE | | |
| (N- | deletion | | | (residues | KALELTGLKVFGNEIKLEKPKGKDSKKERDARTLLAKNLPYKVTQD | | |
| term | | | | 272-710) | ELKEVFEDAAEIRLVSKDGKSKGIAYIEFKTEADAEKTFEEKQGTEI | | |
| del) | | | | | DGRSISLYYTGEKGQNQDYRGGKNSTWSGESKTLVLSNLSYSAT | | |
| | | | | | EETLQEVFEKATFIKVPQNQNGKSKGYAFIEFASFEDAKEALNSCN | | |
| | | | | | KREIEGRAIRLELQGPRGSPNARSQPSKTLFVKGLSEDTTEETLKE | | |
| | | | | | SFDGSVRARIVTDRETGSSKGFGFVDFNSEEDAKAAKEAMEDGEI | | |
| | | | | | DGNKVTLDWAKPKGEGGFGGRGGGRGGFGGRGGGRGGRGGF | | |
| | | | | | GGRGRGGFGGRGGFRGGRGGGGDHKPQGKKTKFE | | |
| NCL3/ | Nucleolin | Homo | NM_00538 | RNA | PVKEAPGKRKKEMAKQKAAPEAKKQKVEGTEPTTAFNLFVGNLN | 29 | 376 |
| NCL | RNA | sapiens | 71 .3 | recogni- | FNKSAPELKTGISDVFAKNDLAVVDVRIGMTRKFGYVDFESAEDLE | | |
| (RRMs) | recognition | | | tion | KALELTGLKVFGNEIKLEKPKGKDSKKERDARTLLAKNLPYKVTQD | | |
| | motifs | | | motifs | ELKEVFEDAAEIRLVSKDGKSKGIAYIEFKTEADAEKTFEEKQGTEI | | |
| | | | | (RRMs) | DGRSISLYYTGEKGQNQDYRGGKNSTWSGESKTLVLSNLSYSAT | | |
| | | | | (residues | EETLQEVFEKATFIKVPQNQNGKSKGYAFIEFASFEDAKEALNSCN | | |
| | | | | 272-647) | KREIEGRAIRLELQGPRGSPNARSQPSKTLFVKGLSEDTTEETLKE | | |
| | | | | | SFDGSVRARIVTDRETGSSKGFGFVDFNSEEDAKAAKEAMEDGEI | | |
| | | | | | DGNKVTLDWAKP | | |
| CNBP | Cellular | Homo | NM_00341 | Full | MSSNECFKCGRSGHWARECPTGGGRGRGMRSRGRGGFTSDR | 30 | 177 |
| | nucleic acid | sapiens | 8.4 | length | GFQFVSSSLPDICYRCGESGHLAKDCDLQEDACYNCGRGGHIAK | | |
| | binding | | | ORF | DCKEPKREREQCCYNCGKPGHLARDCDHADEQKCYSCGEFGHI | | |
| | protein | | | | QKDCTKVKCYRCGETGHVAINCSKTSEVNCYRCGESGHLARECT | | |
| | | | | | IEATA | | |
| IGF2 | Insulin-like | Homo | NM_00061 | Full | MGIPMGKSMLVLLTFLAFASCCIAAYRPSETLCGGELVDTLQFVC | 31 | 180 |
| | growth factor | sapiens | 2.5 | length | GDRGFYFSRPASRVSRRSRGIVEECCFRSCDLALLETYCATPAKS | | |
| | II | | | ORF | ERDVSTPPTVLPDNFPRYPVGKFFQYDTWKQSTQRLRRGLPALL | | |
| | | | | | RARRGHVLAKELEAFREAKRHRPLIALPTQDPAHGGAPPEMASN | | |
| | | | | | RK | | |
| FANCJ | BRCA1-binding | Homo | AF36054 | Full | MSSMWSEYTIGGVKIYFPYKAYPSQLAMMNSILRGLNSKQHCLLE | 32 | 1249 |
| | helicase-like | sapiens | 9.1 | length | SPTGSGKSLALLCSALAWQQSLSGKPADEGVSEKAEVQLSCCCA | | |
| | protein / | | | ORF | CHSKDFTNNDMNQGTSRHFNYPSTPPSERNGTSSTCQDSPEKTT | | |
| | Fanconi | | | | LAAKLSAKKQASIYRDENDDFQVEKKRIRPLETTQQIRKRHCFGTE | | |
| | anemia group | | | | VHNLDAKVDSGKTVKLNSPLEKINSFSPQKPPGHCSRCCCSTKQ | | |
| | J protein | | | | GNSQESSNTIKKDHTGKSKIPKIYFGTRTHKQIAQITRELRRTAYSG | | |
| | | | | | VPMTILSSRDHTCVHPEVVGNFNRNEKCMELLDGKNGKSCYFYH | | |
| | | | | | GVHKISDQHTLQTFQGMCKAWDIEELVSLGKKLKACPYYTARELI | | |
| | | | | | QDADIIFCPYNYLLDAQIRESMDLNLKEQVVILDEAHNIEDCARESA | | |
| | | | | | SYSVTEVQLRFARDELDSMVNNNIRKKDHEPLRAVCCSLINWLEA | | |
| | | | | | NAEYLVERDYESACKIWSGNEMLLTLHKMGITTATFPILQGHFSAV | | |
| | | | | | LQKEEKISPIYGKEEAREVPVISASTQIMLKGLFMVLDYLFRQNSRF | | |
| | | | | | ADDYKIAIQQTYSWTNQIDISDKNGLLVLPKNKKRSRQKTAVHVLN | | |
| | | | | | FWCLNPAVAFSDINGKVQTIVLTSGTLSPMKSFSSELGVTFTIQLE | | |
| | | | | | ANHIIKNSQVWVGTIGSGPKGRNLCATFQNTETFEFQDEVGALLL | | |
| | | | | | SVCQTVSQGILCFLPSYKLLEKLKERVVLSTGLWHNLELVKTVIVEP | | |
| | | | | | QGGEKTNFDELLQVYYDAIKYKGEKDGALLVAVCRGKVSEGLDFS | | |
| | | | | | DDNARAVITIGIPFPNVKDLQVELKRQYNDHHSKLRGLLPGRQWY | | |
| | | | | | EIQAYRALNQALGRCIRHRNDWGALILVDDRFRNNPSRYISGLSK | | |
| | | | | | WVRQQIQHHSTFESALESLAEFSKKHQKVLNVSIKDRTNIQDNES | | |
| | | | | | TLEVTSLKYSTPPYLLEAASHLSPENFVEDEAKICVQELQCPKIITK | | |
| | | | | | NSPLPSSIISRKEKNDPVFLEEAGKAEKIVISRSTSPTFNKQTKRVS | | |
| | | | | | WSSFNSLGQYFTGKIPKATPELGSSENSASSPPRFKTEKMESKTV | | |
| | | | | | LPFTDKCESSNLTVNTSFGSCPQSETIISSLKIDATLTRKNHSEHPL | | |
| | | | | | CSEEALDPDIELSLVSEEDKQSTSNRDFETEAEDESIYFTPELYDP | | |
| | | | | | EDTDEEKNDLAETDRGNRLANNSDCILAKDLFEIRTIKEVDSAREV | | |
| | | | | | KAEDCIDTKLNGILHIEESKIDDIDGNVKTTWINELELGKTHEIEIK | | |
| | | | | | NFKPSPSKNKGMFPGFK | | |
| FANCJ | BRCA1- | Homo | AF36054 | DNA | GGVKIYFPYKAYPSQLAMMNSILRGLNSKQHCLLESPTGSGKSLA | 33 | 432 |
| (DBD) | binding | sapiens | 9.1 | binding | LLCSALAWQQSLSGKPADEGVSEKAEVQLSCCCACHSKDFTNND | | |
| | helicase-like | | | domain | MNQGTSRHFNYPSTPPSERNGTSSTCQDSPEKTTLAAKLSAKKQ | | |
| | protein / | | | (residues | ASIYRDENDDFQVEKKRIRPLETTQQIRKRHCFGTEVHNLDAKVD | | |
| | Fanconi | | | 11-442) | SGKTVKLNSPLEKINSFSPQKPPGHCSRCCCSTKQGNSQESSNTI | | |
| | anemia group | | | | KKDHTGKSKIPKIYFGTRTHKQIAQITRELRRTAYSGVPMTILSSRD | | |
| | J protein DNA | | | | HTCVHPEVVGNFNRNEKCMELLDGKNGKSCYFYHGVHKISDQHT | | |
| | binding | | | | LQTFQGMCKAWDIEELVSLGKKLKACPYYTARELIQDADIIFCPYN | | |
| | domain | | | | YLLDAQIRESMDLNLKEQVVILDEAHNIEDCARESASYSVTEVQLR | | |
| | | | | | FARDELDSMVNNNIRKKDHEPLRAVC | | |
| PIF1 | PIF1 helicase | Homo | NM_00128 | Full | MLSGIEAAAGEYEDSELRCRVAVEELSPGGQPRRRQALRTAELSL | 34 | 641 |
| | | sapiens | 6497.1 | length | GRNERRELMLRLQAPGPAGRPRCFPLRAARLFTRFAEAGRSTLR | | |
| | | | | ORF | LPAHDTPGAGAVQLLLSDCPPDRLRRFLRTLRLKLAAAPGPGPAS | | |
| | | | | | ARAQLLGPRPRDFVTISPVQPEERRLRAATRVPDTTLVKRPVEPQ | | |
| | | | | | AGAEPSTEAPRWPLPVKRLSLPSTKPQLSEEQAAVLRAVLKGQSI | | |
| | | | | | FFTGSAGTGKSYLLKRILGSLPPTGTVATASTGVAACHIGGTTLHA | | |
| | | | | | FAGIGSGQAPLAQCVALAQRPGVRQGWLNCQRLVIDEISMVEADL | | |
| | | | | | FDKLEAVARAVRQQNKPFGGIQLIICGDFLQLPPVTKGSQPPRFCF | | |
| | | | | | QSKSWKRCVPVTLELTKVWRQADQTFISLLQAVRLGRCSDEVTR | | |
| | | | | | QLQATASHKVGRDGIVATRLCTHQDDVALTNERRLQELPGKVHR | | |
| | | | | | FEAMDSNPELASTLDAQCPVSQLLQLKLGAQVMLVKNLSVSRGL | | |
| | | | | | VNGARGVVVGFEAEGRGLPQVRFLCGVTEVIHADRWTVQATGG | | |
| | | | | | QLLSRQQLPLQLAWAMSIHKSQGMTLDCVEISLGRVFASGQAYV | | |
| | | | | | ALSRARSLQGLRVLDFDPMAVRCDPRVLHFYATLRRGRSLSLESP | | |
| | | | | | DDDEAASDQENMDPIL | | |
| BLM | Human | Homo | XM_00672 | Full | MAAVPQNNLQEQLERHSARTLNNKLSLSKPKFSGFTFKKKTSSDN | 35 | 1417 |
| | Bloom's | sapiens | 0632.2 | length | NVSVTNVSVAKTPVLRNKDVNVTEDFSFSEPLPNTTNQQRVKDFF | | |
| | syndrome | | | ORF | KNAPAGQETQRGGSKSLLPDFLQTPKEVVCTTQNTPTVKKSRDT | | |
| | protein | | | | ALKKLEFSSSPDSLSTINDWDDMDDFDTSETSKSFVTPPQSHFVR | | |
| | | | | | VSTAQKSKKGKRNFFKAQLYTTNTVKTDLPPPSSESEQIDLTEEQ | | |
| | | | | | KDDSEWLSSDVICIDDGPIAEVHINEDAQESDSLKTHLEDERDNSE | | |
| | | | | | KKKNLEEAELHSTEKVPCIEFDDDDYDTDFVPPSPEEIISASSSSS | | |
| | | | | | KCLSTLKDLDTSDRKEDVLSTSKDLLSKPEKMSMQELNPETSTDC | | |
| | | | | | DARQISLQQQLIHVMEHICKLIDTIPDDKLKLLDCGNELLQQRNIRR | | |
| | | | | | KLLTEVDFNKSDASLLGSLWRYRPDSLDGPMEGDSCPTGNSMKE | | |
| | | | | | LNFSHLPSNSVSPGDCLLTTTLGKTGFSATRKNLFERPLFNTHLQK | | |
| | | | | | SFVSSNWAETPRLGKKNESSYFPGNVLTSTAVKDQNKHTASINDL | | |
| | | | | | ERETQPSYDIDNFDIDDFDDDDDWEDIMHNLAASKSSTAAYQPIK | | |
| | | | | | EGRPIKSVSERLSSAKTDCLPVSSTAQNINFSESIQNYTDKSAQNL | | |
| | | | | | ASRNLKHERFQSLSFPHTKEMMKIFHKKFGLHNFRTNQLEAINAAL | | |
| | | | | | LGEDCFILMPTGGGKSLCYQLPACVSPGVTVVISPLRSLIVDQVQK | | |
| | | | | | LTSLDIPATYLTGDKTDSEATNIYLQLSKKDPIIKLLYVTPEKICASN | | |
| | | | | | RLISTLENLYERKLLARFVIDEAHCVSQWGHDFRQDYKRMNMLRQ | | |
| | | | | | KFPSVPVMALTATANPRVQKDILTQLKILRPQVFSMSFNRHNLKYY | | |
| | | | | | VLPKKPKKVAFDCLEWIRKHHPYDSGIIYCLSRRECDTMADTLQR | | |
| | | | | | DGLAALAYHAGLSDSARDEVQQKWINQDGCQVICATIAFGMGIDK | | |
| | | | | | PDVRFVIHASLPKSVEGYYQESGRAGRDGEISHCLLFYTYHDVTR | | |
| | | | | | LKRLIMMEKDGNHHTRETHFNNLYSMVHYCENITECRRIQLLAYF | | |
| | | | | | GENGFNPDFCKKHPDVSCDNCCKTKDYKTRDVTDDVKSIVRFVQ | | |
| | | | | | EHSSSQGMRNIKHVGPSGRFTMNMLVDIFLGSKSAKIQSGIFGKG | | |
| | | | | | SAYSRHNAERLFKKLILDKILDEDLYINANDQAIAYVMLGNKAQTVL | | |
| | | | | | NGNLKVDFMETENSSSVKKQKALVAKVSQREEMVKKCLGELTEV | | |
| | | | | | CKSLGKVFGVHYFNIFNTVTLKKLAESLSSDPEVLLQIDGVTEDKL | | |
| | | | | | EKYGAEVISVLQKYSEVVTSPAEDSSPGISLSSSRGPGRSAAEELD | | |
| | | | | | EEIPVSSHYFASKTRNERKRKKMPASQRSKRRKTASSGSKAKGG | | |
| | | | | | SATCRKISSKTKSSSIIGSSSASHTSQATSGANSKLGIMAPPKPINR | | |
| | | | | | PFLKPSYAFS | | |
| BLM | Human | Homo | XM_00672 | DNA | INAALLGEDCFILMPTGGGKSLCYQLPACVSPGVTVVISPLRSLIVD | 36 | 349 |
| (DBD) | Bloom's | sapiens | 0632.2 | binding | QVQKLTSLDIPATYLTGDKTDSEATNIYLQLSKKDPIIKLLYVTPEKI | | |
| | syndrome | | | domain | CASNRLISTLENLYERKLLARFVIDEAHCVSQWGHDFRQDYKRMN | | |
| | protein DNA | | | (residues | MLRQKFPSVPVMALTATANPRVQKDILTQLKILRPQVFSMSFNRH | | |
| | binding | | | 676-1024) | NLKYYVLPKKPKKVAFDCLEWIRKHHPYDSGIIYCLSRRECDTMAD | | |
| | domain | | | | TLQRDGLAALAYHAGLSDSARDEVQQKWINQDGCQVICATIAFGM | | |
| | | | | | GIDKPDVRFVIHASLPKSVEGYYQESGRAGRDGEISHCLLFYTYH | | |
| | | | | | DVTRLKRLIMMEKDGNHHTRETHFNNLY | | |
| DHX36 | DHX36/G4R1/ | Homo | NM_02086 | Full | MSYDYHQNWGRDGGPRSSGGGYGGGPAGGHGGNRGSGGGG | 37 | 1008 |
| | MLEL1 | sapiens | 5.2 | length | GGGGGGRGGRGRHPGHLKGREIGMWYAKKQGQKNKEAERQER | | |
| | | | | ORF | AVVHMDERREEQIVQLLNSVQAKNDKESEAQISWFAPEDHGYGT | | |
| | | | | | EVSTKNTPCSENKLDIQEKKLINQEKKMFRIRNRSYIDRDSEYLLQ | | |
| | | | | | ENEPDGTLDQKLLEDLQKKKNDLRYIEMQHFREKLPSYGMQKEL | | |
| | | | | | VNLIDNHQVTVISGETGCGKTTQVTQFILDNYIERGKGSACRIVCT | | |
| | | | | | QPRRISAISVAERVAAERAESCGSGNSTGYQIRLQSRLPRKQGSIL | | |
| | | | | | YCTTGIILQWLQSDPYLSSVSHIVLDEIHERNLQSDVLMTVVKDLLN | | |
| | | | | | FRSDLKVILMSATLNAEKFSEYFGNCPMIHIPGFTFPVVEYLLEDVI | | |
| | | | | | EKIRYVPEQKEHRSQFKRGFMQGHVNRQEKEEKEAIYKERWPDY | | |
| | | | | | VRELRRRYSASTVDVIEMMEDDKVDLNLIVALIRYIVLEEEDGAILV | | |
| | | | | | FLPGWDNISTLHDLLMSQVMFKSDKFLIIPLHSLMPTVNQTQVFKR | | |
| | | | | | TPPGVRKIVIATNIAETSITIDDVVYVIDGGKIKETHFDTQNNISTMSA | | |
| | | | | | EWVSKANAKQRKGRAGRVQPGHCYHLYNGLRASLLDDYQLPEIL | | |
| | | | | | RTPLEELCLQIKILRLGGIAYFLSRLMDPPSNEAVLLSIRHLMELNAL | | |
| | | | | | DKQEELTPLGVHLARLPVEPHIGKMILFGALFCCLDPVLTIAASLSF | | |
| | | | | | KDPFVIPLGKEKIADARRKELAKDTRSDHLTVVNAFEGWEEARRR | | |
| | | | | | GFRYEKDYCVVEYFLSSNTLQMLHNMKGQFAEHLLGAGFVSSRN | | |
| | | | | | PKDPESNINSDNEKIIKAVICAGLYPKVAKIRLNLGKKRKMVKVYTK | | |
| | | | | | TDGLVAVHPKSVNVEQTDFHYNWLIYHLKMRTSSIYLYDCTEVSP | | |
| | | | | | YCLLFFGGDISIQKDNDQETIAVDEWIVFQSPARIAHLVKELRKELD | | |
| | | | | | ILLQEKIESPHPVDWNDTKSRDCAVLSAIIDLIKTQEKATPRNFPPR | | |
| | | | | | FQDGYYS | | |
| DHX36 | DHX36 | Homo | NM_02086 | RHAU- | MSYDYHQNWGRDGGPRSSGGGYGGGPAGGHGGNRGSGGGG | 38 | 157 |
| (G4 | /G4R1/MLEL1 | sapiens | 5.2 | specific | GGGGGGRGGRGRHPGHLKGREIGMWYAKKQGQKNKEAERQER | | |
| BD) | RHAU-specific | | | motif | AVVHMDERREEQIVQLLNSVQAKNDKESEAQISWFAPEDHGYGT | | |
| | motif | | | (RSM) | EVSTKNTPCSENKLDIQEKKLINQEKKMFRI | | |
| | | | | of DHX36 | | | |
| | | | | (residues | | | |
| | | | | 1-157) | | | |
| WRN | Werner | Homo | XM_01154 | Full | MSEKKLETTAQQRKCPEWMNVQNKRCAVEERKACVRKSVFEDD | 39 | 1432 |
| | syndrome | sapiens | 4639.2 | length | LPFLEFTGSIVYSYDASDCSFLSEDISMSLSDGDVVGFDMEWPPL | | |
| | RecQ like | | | ORF | YNRGKLGKVALIQLCVSESKCYLFHVSSMSVFPQGLKMLLENKAV | | |
| | helicase | | | | KKAGVGIEGDQVVKLLRDFDIKLKNFVELTDVANKKLKCTETWSLN | | |
| | | | | | SLVKHLLGKQLLKDKSIRCSNWSKFPLTEDQKLYAATDAYAGFIIY | | |
| | | | | | RNLEILDDTVQRFAINKEEEILLSDMNKQLTSISEEVMDLAKHLPHA | | |
| | | | | | FSKLENPRRVSILLKDISENLYSLRRMIIGSTNIETELRPSNNLNLLS | | |
| | | | | | FEDSTTGGVQQKQIREHEVLIHVEDETWDPTLDHLAKHDGEDVLG | | |
| | | | | | NKVERKEDGFEDGVEDNKLKENMERACLMSLDITEHELQILEQQS | | |
| | | | | | QEEYLSDIAYKSTEHLSPNDNENDTSYVIESDEDLEMEMLKHLSP | | |
| | | | | | NDNENDTSYVIESDEDLEMEMLKSLENLNSGTVEPTHSKCLKMER | | |
| | | | | | NLGLPTKEEEEDDENEANEGEEDDDKDFLWPAPNEEQVTCLKMY | | |
| | | | | | FGHSSFKPVQWKVIHSVLEERRDNVAVMATGYGKSLCFQYPPVY | | |
| | | | | | VGKIGLVISPLISLMEDQVLQLKMSNIPACFLGSAQSENVLTDIKLG | | |
| | | | | | KYRIVYVTPEYCSGNMGLLQQLEADIGITLIAVDEAHCISEWGHDF | | |
| | | | | | RDSFRKLGSLKTALPMVPIVALTATASSSIREDIVRCLNLRNPQITC | | |
| | | | | | TGFDRPNLYLEVRRKTGNILQDLQPFLVKTSSHWEFEGPTIIYCPS | | |
| | | | | | RKMTQQVTGELRKLNLSCGTYHAGMSFSTRKDIHHRFVRDEIQC | | |
| | | | | | VIATIAFGMGINKADIRQVIHYGAPKDMESYYQEIGRAGRDGLQSS | | |
| | | | | | CHVLWAPADINLNRHLLTEIRNEKFRLYKLKMMAKMEKYLHSSRC | | |
| | | | | | RRQIILSHFEDKQVQKASLGIMGTEKCCDNCRSRLDHCYSMDDSE | | |
| | | | | | DTSWDFGPQAFKLLSAVDILGEKFGIGLPILFLRGSNSQRLADQYR | | |
| | | | | | RHSLFGTGKDQTESWWKAFSRQLITEGFLVEVSRYNKFMKICALT | | |
| | | | | | KKGRNWLHKANTESQSLILQANEELCPKKLLLPSSKTVSSGTKEH | | |
| | | | | | CYNQVPVELSTEKKSNLEKLYSYKPCDKISSGSNISKKSIMVQSPE | | |
| | | | | | KAYSSSQPVISAQEQETQIVLYGKLVEARQKHANKMDVPPAILATN | | |
| | | | | | KILVDMAKMRPTTVENVKRIDGVSEGKAAMLAPLLEVIKHFCQTNS | | |
| | | | | | VQTDLFSSTKPQEEQKTSLVAKNKICTLSQSMAITYSLFQEKKMPL | | |
| | | | | | KSIAESRILPLMTIGMHLSQAVKAGCPLDLERAGLTPEVQKIIADVIR | | |
| | | | | | NPPVNSDMSKISLIRMLVPENIDTYLIHMAIEILKHGPDSGLQPSCD | | |
| | | | | | VNKRRCFPGSEEICSSSKRSKEEVGINTETSSAERKRRLPVWFAK | | |
| | | | | | GSDTSKKLMDKTKRGGLFS | | |
| WRN | Werner | Homo | XM_01154 | DNA | HSVLEERRDNVAVMATGYGKSLCFQYPPVYVGKIGLVISPLISLME | 40 | 436 |
| (DBD) | syndrome | sapiens | 4639.2 | binding | DQVLQLKMSNIPACFLGSAQSENVLTDIKLGKYRIVYVTPEYCSGN | | |
| | RecQ like | | | domain | MGLLQQLEADIGITLIAVDEAHCISEWGHDFRDSFRKLGSLKTALP | | |
| | helicase DNA | | | (residues | MVPIVALTATASSSIREDIVRCLNLRNPQITCTGFDRPNLYLEVRRK | | |
| | binding | | | 558-993) | TGNILQDLQPFLVKTSSHWEFEGPTIIYCPSRKMTQQVTGELRKLN | | |
| | domain | | | | LSCGTYHAGMSFSTRKDIHHRFVRDEIQCVIATIAFGMGINKADIR | | |
| | | | | | QVIHYGAPKDMESYYQEIGRAGRDGLQSSCHVLWAPADINLNRH | | |
| | | | | | LLTEIRNEKFRLYKLKMMAKMEKYLHSSRCRRQIILSHFEDKQVQK | | |
| | | | | | ASLGIMGTEKCCDNCRSRLDHCYSMDDSEDTSWDFGPQAFKLLS | | |
| | | | | | AVDILGEKFGIGLPILFLRGSNSQR | | |
| DHX36 | DEAH/RHA | Bos | PDB: | Has N- | GHPGHLKGREIGLWYAKKQGQKNKEAERQERAVVHMDERREEQ | 41 | 930 |
| | helicase | taurus | 5VHA | terminal | IVQLLHSVQTKNDKDEEAQISWFAPEDHGYGTEAYIDRDSEYLLQ | | |
| | DHX36 | | | trunca- | ENEPDATLDQQLLEDLQKKKTDLRYIEMQRFREKLPSYGMQKELV | | |
| | | | | tion, | NMIDNHQVTVISGETGCGKTTQVTQFILDNYIERGKGSACRIVCTQ | | |
| | | | | sequence | PRRISAISVAERVAAERAESCGNGNSTGYQIRLQSRLPRKQGSILY | | |
| | | | | used for | CTTGIILQWLQSDPHLSSVSHIVLDEIHERLQSDVLMTVVKDLLSYR | | |
| | | | | structure | PDLKVVLMSATLNAEKFSEYFGNCPMIHIPGFTFPVVEYLLEDIIEKI | | |
| | | | | determi- | RYVPEQKEHRSQFKKGFMQGHVNRQEKYYYEAIYKERWPGYLR | | |
| | | | | nation | ELRQRYSASTVDVVEMMDDEKVDLNLIAALIRYIVLEEEDGAILVFL | | |
| | | | | (PMID: | PGWDNISTLHDLLMSQVMFKSDKFIIIPLHSLMPTVNQTQVFKRTP | | |
| | | | | 29899445) | PGVRKIVIATNIAETSITIDDVVYVIDGGKIKETHFDTQNNISTMSAE | | |
| | | | | | WVSKANKQRKGRAGRVQPGHCYHLYNSLRASLLDDYQLPEILRT | | |
| | | | | | PLEELCLQIKILRLGGIAHFLSRLMDPPSNEAVLLSIKHLMELNALD | | |
| | | | | | KQEELTPLGVHLARLPVEPHIGKMILFGALFCCLDPVLTIAASLSFK | | |
| | | | | | DPFVIPLGKEKVADARRKELAAATASDHLTVVNAFKGWEKAKQRG | | |
| | | | | | FRYEKDYCWEYFLSSNTLQMLHNMKGQFAEHLLGAGFVSSRNP | | |
| | | | | | QDPESNINSDNEKIIKAVICAGLYPKVAKIRLNLGKRKMVKVYTKTD | | |
| | | | | | GVVAIHPKSVNVEQTEFNYNWLIYHLKMRTSSIYLYDCTEVSPYCL | | |
| | | | | | LFFGGDISIQKDNDQETIAVDEWIIFQSPARIAHLVKELRKELDILLQ | | |
| | | | | | EKIESPHPVDVVKDTKSRDCAVLSAIIDLIKTQEKATPRNLPPRFQD | | |
| | | | | | GYYSPHHHHHHHH | | |

### Binding Experiments.

Microarrays were preincubated with a 100 mM potassium chloride solution for 1 h at RT to induce G4 formation. Protein binding microarray experiments were then performed as previously described.²³ Microarrays were blocked with 4% nonfat dry milk in a potassium phosphate buffer before incubation with proteins or small molecules. Expressed proteins were blocked with 4% nonfat dry milk, ssDNA, and BSA. For the validation experiments, microarrays were also treated with 100 mM lithium chloride to inhibit G4 formation. For experiments examining dsDNA, single-stranded DNA probes were made double-stranded using a primer complementary to a 24-mer constant sequence following the method described previously.^{23,24} Double stranding efficiency was monitored using 4% Cy3-dCTP.

### Data Processing and Analysis.

Protein or molecule-bound microarrays were scanned with the G5761A SureScan Dx Microarray Scanner System (Agilent) to detect a Cy5 signal at two laser settings (30 and 100 PMT) to ensure signal intensities were below saturation. Spot intensities from microarray images were extracted using the Agilent Feature Extraction Software and are reported as raw fluorescence units. All binding assays were performed at least twice, with high agreement between replicates (R>0.8). Microarrays with the fewest number of saturated spots were used for further analysis. Median intensity was then computed for probes containing identical sequence on each microarray. Sequence logos were generated from a position frequency matrix generated from selected sequences using ggseqlogo.⁴⁸ To gauge the correlation between G4-seq and the microarray data, G4detector⁴² with a pretrained model on human genomic G4s stabilized by K+ and PDS with randomized negative genomic sequence was used.²⁸ For each microarray probe sequence, G4detector was used to predict the probability of it being a G4, i.e., a number between 0 and 1. The measured array data (Design 3, PDS) and predictions were normalized using the following

Y=log(1−Xᵢ−min(X)) (1)

where X is the vector of array intensity measurements or G4 probability predictions. The Pearson correlation between log normalized predicted probabilities and log normalized intensities is reported.

The following clauses show several illustrative and non-limiting embodiments of the invention:

1. A method for determining binding preferences of a non-fluorescent test compound for one or more target G-quadruplex moieties, the method comprising;

a) incubating a device comprising a plurality of single-stranded nucleic acid molecules capable of forming one or more G-quadruplex moieties including the target G-quadruplex moieties with a solution comprising a G-quadruplex stabilizing cation selected from the group consisting of Na⁺ and K⁺;

b) incubating the device with a solution of a compound capable of providing a fluorescent signal (a fluorescent compound), wherein the fluorescent compound is capable of binding to the target G-quadruplex moieties;

c) measuring a first fluorescent signal from the fluorescent compound bound to the device;

d) removing the fluorescent compound from the device;

e) contacting the device with a solution of the fluorescent compound and the test compound;

f) measuring a second fluorescent signal from the fluorescent compound bound to the device; and

g) using the first fluorescent signal and the second fluorescent signal to calculate the binding preferences of the test compound.

2. The method of clause 1 wherein the device is a microarray comprising a plurality of single-stranded DNA molecules (s-DNAs) attached to a solid substrate; where

each s-DNA is from 50 nucleotides (nt) to 100 nt in length and

includes an independently selected linker sequence and an independently selected G-quadruplex-forming region (G4 sequence) where the G4 sequence has formula I

S1-T1-S2-T2-S3-T3-S4-T4-S5 (I) (SEQ ID NO: 54)

wherein T1 is G-Gx1, T2 is G-Gx2, T3 is G-Gx3, and T4 is G-Gx4;

S1 to S5 are independently selected sequences of from 0 to 5 nucleotides independently selected in each instance from the group consisting of A, T, C, and G; and

x1 to x4 are each independently selected in each instance from the group consisting of 2, 3, 4, and 5.

3. The method of clause 2 wherein the G-quadruplex stabilizing cation is K⁺.

4. The method of clause 2 wherein the G4 sequence is selected from the group consisting of

(SEQ ID NO: 42)
5′-TTATGGGGAGGGTGGGGAGGGTGGGGAAGGTGGGGAGGAG-3′,
(SEQ ID NO: 43)
5′-TTGGGGAGGGTGGGGAGGGTGGGGAAGGT-3′,
(SEQ ID NO: 10)
5′-TGGGGAGGGTGGGGAGGGTGGGGAAGG-3′,
(SEQ ID NO: 9)
5′-TTGGGGAGGGTGGGGAGGGTGGGGAA-3′,
(SEQ ID NO: 6)
5′-TGAGGGTGGGGAGGGTGGGGAA-3′,
(SEQ ID NO: 4)
5′-TGAGGGTGGGTAGGGTGGGTAA-3′,
(SEQ ID NO: 7)
5′-AGGGTGGGGAGGGTGGGG-3′,
(SEQ ID NO: 44)
5′-GCTGGGAGAAGGGGGGGCGGCGGGGCAGGGAGGGTGGACGC-3′,
(SEQ ID NO: 45)
5′-TTGGGAGAAGGGGGGGCGGCGGGGCA-3′,
(SEQ ID NO: 46)
5′-AAGGGAGGGCGGCGGGGCA-3′,
(SEQ ID NO: 47)
5′-AAGGGGGGGCGGCGGGGCAGGGAGGGT-3′,
(SEQ ID NO: 26)
5′-CGGCGGGGCAGGGAGGGTGGACG-3′,
(SEQ ID NO: 48)
5′-AGGGTTAGGGTTAGGGTTAGGG-3′,
(SEQ ID NO: 49)
5′-TTAGGGTTAGGGTTAGGGTTAGGGAAA-3′,
(SEQ ID NO: 50)
5′-TTAGGGTTAGGGTTAGGGTTAGGGTTA-3′,
(SEQ ID NO: 17)
5′-AGGGGCGGGCGCGGGAGGAAGGGGGCGGGA-3′,
(SEQ ID NO: 18)
5′-CGGGCGGGAGCGCGGCGGGCGGGCGGGC-3′,
(SEQ ID NO: 24)
5′-GGAGGCGGGGGGGGGGGGGCGGGGGCGGGGGCGGGGGAGGGGCG
CGGC-3′,
(SEQ ID NO: 12)
5′-AGGGCGGTGTGGGAAGAGGGAAGAGGGGGAGGCAG-3′,
(SEQ ID NO: 13)
5′-AGGGCGGTGTGGGAATAGGGAA-3′,
(SEQ ID NO: 15)
5′-CGGGGCGGGCCGGGGGCGGGGT-3′,
(SEQ ID NO: 23)
5′-GGGTAGGGGCGGGGCGGGGCGGGGGC-3′,
(SEQ ID NO: 20)
5′-GGAGGAGGAGGTCACGGAGGAGGAGGAGAAGGAGGAGGAGGA-3′,
(SEQ ID NO: 19)
5′-GGGAGGGAGAGGGGGCGGG-3′,
and,
(SEQ ID NO: 16)
5′-AGGGAGGGCGCTGGGAGGAGGG-3′.

5. The method of clause 2 wherein the G4 sequence is

(SEQ ID NO: 51)
5′-TGA₁₋₅GGGT₁₋₅GGG(GA)₁₋₅GGGT₁₋₅GGGGAA-3′,
or
(SEQ ID NO: 52)
5′-TGA₁₋₅GGGA₁₋₅GGGA₁₋₅GGGA₁₋₅GGGGAA-3′

6. The method of clause 2 wherein the G4 sequence is 5′-NNGGGTGGGGAGGGTGGGNN-3′ (SEQ ID NO: 3), where each N is independently selected in each instance from the group consisting of A, T, C, and G.

7. The method of clause 2 wherein the G4 sequence occurs in a human oncogene.

8. The method of clause 2 wherein the test compound is a protein, an oligopeptide, an oligonucleotide, or a small molecule.

9. The method of clause 8 wherein the test compound is a protein.

10. The method of clause 8 wherein the test compound is a small molecule.

11. A method for determining the binding preference of a test compound capable of providing a fluorescent signal (a fluorescent test compound) for one or more target G-quadruplex moieties, the method comprising the steps of;

a) incubating a device comprising a plurality of single-stranded nucleic acid molecules capable of forming one or more G-quadruplex moieties including the target G-quadruplex moieties with a solution comprising a G-quadruplex stabilizing cation selected from the group consisting of Na⁺ and K⁺;

b) contacting the fluorescent test compound with the device;

c) measuring a first fluorescent signal from the fluorescent test compound bound to the device;

d) incubating the device with a solution of solution of Li+;

e) contacting the fluorescent test compound with the device;

f) measuring a second fluorescent signal from the fluorescent test compound bound to the device;

g) using the first fluorescent signal and the second fluorescent signal to calculate the binding preference of the fluorescent test compound.

12. The method of clause 11 wherein the device is a microarray comprising a plurality of single-stranded DNA molecules (s-DNAs) attached to a solid substrate; where

each s-DNA is from 50 nt to 100 nt in length and

includes an independently selected linker sequence and an independently selected G-quadruplex-forming region (G4 sequence) where the G4 sequence has formula I

S1-T1-S2-T2-S3-T3-S4-T4-S5 (I) (SEQ ID NO: 54)

wherein T1 is G-Gx1, T2 is G-Gx2, T3 is G-Gx3, and T4 is G-Gx4;

S1 to S5 are independently selected sequences of from 0 to 5 nucleotides independently selected in each instance from the group consisting of A, T, C, and G; and

x1 to x4 are each independently selected from the group consisting of 2, 3, 4, and 5.

13. The method of clause 12 wherein the G-quadruplex stabilizing cation is K⁺.

14. The method of clause 12 wherein the G4 sequence is selected from the group consisting of

(SEQ ID NO: 42)
5′-TTATGGGGAGGGTGGGGAGGGTGGGGAAGGTGGGGAGGAG-3′,
(SEQ ID NO: 43)
5′-TTGGGGAGGGTGGGGAGGGTGGGGAAGGT-3′,
(SEQ ID NO: 10)
5′-TGGGGAGGGTGGGGAGGGTGGGGAAGG-3′,
(SEQ ID NO: 9)
5′-TTGGGGAGGGTGGGGAGGGTGGGGAA-3′,
(SEQ ID NO: 6)
5′-TGAGGGTGGGGAGGGTGGGGAA-3′,
(SEQ ID NO: 4)
5′-TGAGGGTGGGTAGGGTGGGTAA-3′,
(SEQ ID NO: 7)
5′-AGGGTGGGGAGGGTGGGG-3′,
(SEQ ID NO: 44)
5′-GCTGGGAGAAGGGGGGGCGGCGGGGCAGGGAGGGTGGACGC-3′,
(SEQ ID NO: 45)
5′-TTGGGAGAAGGGGGGGCGGCGGGGCA-3′,
(SEQ ID NO: 46)
5′-AAGGGAGGGCGGCGGGGCA-3′,
(SEQ ID NO: 47)
5′-AAGGGGGGGCGGCGGGGCAGGGAGGGT-3′,
(SEQ ID NO: 26)
5′-CGGCGGGGCAGGGAGGGTGGACG-3′,
(SEQ ID NO: 48)
5′-AGGGTTAGGGTTAGGGTTAGGG-3′,
(SEQ ID NO: 49)
5′-TTAGGGTTAGGGTTAGGGTTAGGGAAA-3′,
(SEQ ID NO: 50)
5′-TTAGGGTTAGGGTTAGGGTTAGGGTTA-3′,
(SEQ ID NO: 17)
5′-AGGGGCGGGCGCGGGAGGAAGGGGGCGGGA-3′,
(SEQ ID NO: 18)
5′-CGGGCGGGAGCGCGGCGGGCGGGCGGGC-3′,
(SEQ ID NO: 24)
5′-GGAGGCGGGGGGGGGGGGGCGGGGGCGGGGGCGGGGGAGGGGCG
CGGC-3′,
(SEQ ID NO: 12)
5′-AGGGCGGTGTGGGAAGAGGGAAGAGGGGGAGGCAG-3′,
(SEQ ID NO: 13)
5′-AGGGCGGTGTGGGAATAGGGAA-3′,
(SEQ ID NO: 15)
5′-CGGGGCGGGCCGGGGGCGGGGT-3′,
(SEQ ID NO: 23)
5′-GGGTAGGGGCGGGGCGGGGCGGGGGC-3′,
(SEQ ID NO: 20)
5′-GGAGGAGGAGGTCACGGAGGAGGAGGAGAAGGAGGAGGAGGA-3′,
(SEQ ID NO: 19)
5′-GGGAGGGAGAGGGGGCGGG-3′,
and,
(SEQ ID NO: 16)
5′-AGGGAGGGCGCTGGGAGGAGGG-3′.

15. The method of clause 12 wherein the G4 sequence is

(SEQ ID NO: 51)
5′-TGA₁₋₅GGGT₁₋₅GGG(GA)₁₋₅GGGT₁₋₅GGGGAA-3′,
or
(SEQ ID NO: 52)
5′-TGA₁₋₅GGGA₁₋₅GGGA₁₋₅GGGA₁₋₅GGGGAA-3′

16 The method of clause 12 wherein the G4 sequence is

(SEQ ID NO: 3)
5′-NNGGGTGGGGAGGGTGGGNN-3′

where each N is independently selected in each instance from the group consisting of A, T, C, and G.

17. The method of clause 12 wherein the G4 sequence occurs in a human oncogene.

18. The method of clause 12 wherein the test compound is a protein, an oligopeptide, an oligonucleotide, or a small molecule.

19. The method of clause 12 wherein the test compound is a protein.

20. The method of clause 12 wherein the test compound is a small molecule.

In another embodiment, the one or more targeted G4-quadruplex moieties occur in one or single-stranded oligonucleotides (single-stranded DNA or RNA molecules).

In another embodiment, the one or more targeted G4-quadruplex moieties occur in one or single-stranded oligonucleotides (single-stranded DNA or RNA molecules) containing one or more chemically modified nucleotides.

In another embodiment, the device is a microarray comprising a plurality of single-stranded DNA or RNA molecules (s-DNAs or RNAs) attached to a solid substrate; where each s-DNA or RNA is from 50 nucleotides (nt) to 100 nt in length and includes an independently selected linker sequence and an independently selected G-quadruplex-forming region (G4 sequence) where the G4 sequence has formula II, S1-T1-S2-T2-S3-T3-S4-T4-S5 (II) (SEQ ID NO: 55), wherein T1 is G-Gx1, T2 is G-Gx2, T3 is G-Gx3, and T4 is G-Gx4; S1 to S5 are independently selected sequences of from 0 to 4 nucleotides independently selected in each instance from the group consisting of A, T, U, C, and G; and x1 to x4 are each independently selected in each instance from the group consisting of 2, 3, 4, and 5.

In another embodiment, the one or more targeted G4-quadruplex moieties occur in one or more nucleic acid aptamers. Aptamers are short single-stranded oligonucleotides (single-stranded DNA or RNA molecules) that are capable of binding various target molecules with high affinity and specificity. The DNA or RNA molecules in the aptamer may contained one or more chemically modified nucleotide. It has been found that many aptamers are capable of forming G4-quadruplex moieties.

In another embodiment, the G4-quadruplex moiety is formed in a single-stranded oligonucleotide molecule containing chemically modified nucleotides. In a non-limiting example the single-stranded oligonucleotide molecule containing the G4-quadruplex includes one or more nucleotides modified at the 2′-position of the ribose portion of the nucleotide. The 2′-fluoro (2′-F), 2′-amino (2′-NH2) and 2′-O-methyl (2′-OMe) are common 2′-substituent modifications on the ribose unit. These modifications may increase nuclease resistance and/or optimize aptamer affinity for its target molecules.

In another embodiment, the method of any one of the preceding embodiments wherein the test compound or the fluorescent test compound is independently a protein, an oligopeptide, an oligonucleotide, or a small molecule.

The term “small molecule” as used herein, generally refers to an organic chemical compound of less than about 1,000 Da

The terms “G4 sequences” and “G4-forming sequences” are and can be used interchangeably herein and generally refer to sequences capable of forming G quadruplexes.

### REFERENCES FOR PART A

(1) Weirauch, M. T., Yang, A., Albu, M., Cote, A. G., Montenegro-Montero, A., Drewe, P., Najafabadi, H. S., Lambert, S. A., Mann, I., Cook, K., Zheng, H., Goity, A., van Bakel, H., Lozano, J. C., Galli, M., Lewsey, M. G., Huang, E., Mukherjee, T., Chen, X., Reece-Hoyes, J. S., Govindarajan, S., Shaulsky, G., Walhout, A. J. M., Bouget, F. Y., Ratsch, G., Larrondo, L. F., Ecker, J. R., and Hughes, T. R. (2014) Determination and inference of eukaryotic transcription factor sequence specificity. Cell 158, 1431-1443.
(2) Guiblet, W. M., Cremona, M. A., Cechova, M., Harris, R. S., Kejnovska, I., Kejnovsky, E., Eckert, K., Chiaromonte, F., and Makova, K. D. (2018) Long-read sequencing technology indicates genomewide effects of non-B DNA on polymerization speed and error rate. Genome Res. 28, 1767-1778.
(3) Ashton, N. W., Bolderson, E., Cubeddu, L., O'Byrne, K. J., and Richard, D. J. (2013) Human single-stranded DNA binding proteins are essential for maintaining genomic stability. BMC Mol. Biol. 14, 9.
(4) Mishra, S. K., Tawani, A., Mishra, A., and Kumar, A. (2016) G4IPDB: A database for G-quadruplex structure forming nucleic acid interacting proteins. Sci. Rep. 6, 38144.
(5) Gellert, M., Lipsett, M. N., and Davies, D. R. (1962) Helix formation by guanylic acid. Proc. Natl. Acad. Sci. U.S.A 48, 2013-2018.
(6) Rhodes, D., and Lipps, H. J. (2015) G-quadruplexes and their regulatory roles in biology. Nucleic Acids Res. 43, 8627-8637.
(7) Hansel-Hertsch, R., Di Antonio, M., and Balasubramanian, S. (2017) DNA G-quadruplexes in the human genome: detection, functions and therapeutic potential. Nat. Rev. Mol. Cell Biol. 18, 279-284.
(8) Konig, S. L., Evans, A. C., and Huppert, J. L. (2010) Seven essential questions on G-quadruplexes. Biomol. Concepts 1, 197-213. (9) Siddiqui-Jain, A., Grand, C. L., Bearss, D. J., and Hurley, L. H. (2002) Direct evidence for a G-quadruplex in a promoter region and its targeting with a small molecule to repress c-MYC transcription. Proc. Natl. Acad. Sci. U.S.A 99, 11593-11598.
(10) Dai, J., Dexheimer, T. S., Chen, D., Carver, M., Ambrus, A., Jones, R. A., and Yang, D. (2006) An intramolecular G-quadruplex structure with mixed parallel/antiparallel G-strands formed in the human BCL-2 promoter region in solution. J. Am. Chem. Soc. 128, 1096-1098.
(11) Yang, D., and Okamoto, K. (2010) Structural insights into Gquadruplexes: towards new anticancer drugs. Future Med. Chem. 2, 619-646. (12) Neidle, S. (2016) Quadruplex Nucleic Acids as Novel Therapeutic Targets. J. Med. Chem. 59, 5987-6011.
(13) Rodriguez, R., Muller, S., Yeoman, J. A., Trentesaux, C., Riou, J. F., and Balasubramanian, S. (2008) A novel small molecule that alters shelterin integrity and triggers a DNA-damage response at telomeres. J. Am. Chem. Soc. 130, 15758-15759.
(14) Parkinson, G. N., Ghosh, R., and Neidle, S. (2007) Structural basis for binding of porphyrin to human telomeres. Biochemistry 46, 2390-2397.
(15) Calabrese, D. R., Chen, X., Leon, E. C., Gaikwad, S. M., Phyo, Z., Hewitt, W. M., Alden, S., Hilimire, T. A., He, F., Michalowski, A. M., Simmons, J. K., Saunders, L. B., Zhang, S., Connors, D., Walters, K. J., Mock, B. A., and Schneekloth, J. S., Jr. (2018) Chemical and structural studies provide a mechanistic basis for recognition of the MYC G-quadruplex. Nat. Commun. 9, 4229.
(16) Mendoza, O., Bourdoncle, A., Boule, J. B., Brosh, R. M., Jr., and Mergny, J. L. (2016) G-quadruplexes and helicases. Nucleic Acids Res. 44, 1989-2006.
(17) Gonzalez, V., Guo, K., Hurley, L., and Sun, D. (2009) Identification and characterization of nucleolin as a c-myc G quadruplex-binding protein. J. Biol. Chem. 284, 23622-23635.
(18) Connor, A. C., Frederick, K. A., Morgan, E. J., and McGown, L. B. (2006) Insulin capture by an insulin-linked polymorphic region G-quadruplex DNA oligonucleotide. J. Am. Chem. Soc. 128, 4986-4991.
(19) Armas, P., Nasif, S., and Calcaterra, N. B. (2008) Cellular nucleic acid binding protein binds G-rich single-stranded nucleic acids and may function as a nucleic acid chaperone. J. Cell. Biochem. 103, 1013-1036.
(20) Kouzine, F., Wojtowicz, D., Baranello, L., Yamane, A., Nelson, S., Resch, W., Kieffer-Kwon, K. R., Benham, C. J., Casellas, R., Przytycka, T. M., and Levens, D. (2017) Permanganate/S1 Nuclease Footprinting Reveals Non-B DNA Structures with Regulatory Potential across a Mammalian Genome. Cell Syst 4, 344-356.e347.
(21) Mao, S. Q., Ghanbarian, A. T., Spiegel, J., Martinez Cuesta, S., Beraldi, D., Di Antonio, M., Marsico, G., Hansel-Hertsch, R., Tannahill, D., and Balasubramanian, S. (2018) DNA G-quadruplex structures mold the DNA methylome. Nat. Struct. Mol. Biol. 25, 951-957.
(22) Stewart, A. J., Hannenhalli, S., and Plotkin, J. B. (2012) Why transcription factor binding sites are ten nucleotides long. Genetics 192, 973-985.
(23) Badis, G., Berger, M. F., Philippakis, A. A., Talukder, S., Gehrke, A. R., Jaeger, S. A., Chan, E. T., Metzler, G., Vedenko, A., Chen, X., Kuznetsov, H., Wang, C. F., Coburn, D., Newburger, D. E., Morris, Q., Hughes, T. R., and Bulyk, M. L. (2009) Diversity and complexity in DNA recognition by transcription factors. Science 324, 1720-1723.
(24) Berger, M. F., and Bulyk, M. L. (2009) Universal protein binding microarrays for the comprehensive characterization of the DNA-binding specificities of transcription factors. Nat. Protoc. 4, 393-411.
(25) Varizhuk, A., Ilyinsky, N., Smirnov, I., and Pozmogova, G. (2016) G4 Aptamers: Trends in Structural Design. Mini-Rev. Med. Chem. 16, 1321-1329.
(26) Iida, K., Nakamura, T., Yoshida, W., Tera, M., Nakabayashi, K., Hata, K., Ikebukuro, K., and Nagasawa, K. (2013) Fluorescent-ligand mediated screening of G-quadruplex structures using a DNA microarray. Angew. Chem., Int. Ed. 52, 12052-12055.
(27) Weitzmann, M. N., Woodford, K. J., and Usdin, K. (1996) The development and use of a DNA polymerase arrest assay for the evaluation of parameters affecting intrastrand tetraplex formation. J. Biol. Chem. 271, 20958-20964.
(28) Chambers, V. S., Marsico, G., Boutell, J. M., Di Antonio, M., Smith, G. P., and Balasubramanian, S. (2015) High-throughput sequencing of DNA G-quadruplex structures in the human genome. Nat. Biotechnol. 33, 877-881.
(29) Andrilenas, K. K., Penvose, A., and Siggers, T. (2015) Using protein-binding microarrays to study transcription factor specificity: homologs, isoforms and complexes. Briefings Funct. Genomics 14, 17-29.
(30) Bedrat, A., Lacroix, L., and Mergny, J. L. (2016) Re-evaluation of G-quadruplex propensity with G4Hunter. Nucleic Acids Res. 44, 1746-1759.
(31) Biffi, G., Tannahill, D., McCafferty, J., and Balasubramanian, S. (2013) Quantitative visualization of DNA G-quadruplex structures in human cells. Nat. Chem. 5, 182-186.
(32) Bhattacharyya, D., Mirihana Arachchilage, G., and Basu, S. (2016) Metal Cations in G-Quadruplex Folding and Stability. Front. Chem. 4, 38.
(33) Khund-Sayeed, S., He, X., Holzberg, T., Wang, J., Rajagopal, D., Upadhyay, S., Durell, S. R., Mukherjee, S., Weirauch, M. T., Rose, R., and Vinson, C. (2016) 5-Hydroxymethylcytosine in E-box motifs ACAT|GTG and ACAC|GTG increases DNA-binding of the B-HLH transcription factor TCF4. Integr Biol. (Camb) 8, 936-945.
(34) Adrian, M., Ang, D. J., Lech, C. J., Heddi, B., Nicolas, A., and Phan, A. T. (2014) Structure and conformational dynamics of a stacked dimeric G-quadruplex formed by the human CEB1 minisatellite. J. Am. Chem. Soc. 136, 6297-6305.
(35) Ambrus, A., Chen, D., Dai, J., Jones, R. A., and Yang, D. (2005) Solution structure of the biologically relevant G-quadruplex element in the human c-MYC promoter. Implications for G-quadruplex stabilization. Biochemistry 44, 2048-2058.
(36) Dai, J., Carver, M., Hurley, L. H., and Yang, D. (2011) Solution structure of a 2:1 quindoline-c-MYC G-quadruplex: insights into G-quadruplex-interactive small molecule drug design. J. Am. Chem. Soc. 133, 17673-17680.
(37) Onel, B., Carver, M., Wu, G., Timonina, D., Kalarn, S., Larriva, M., and Yang, D. (2016) A New G-Quadruplex with Hairpin Loop Immediately Upstream of the Human BCL2 P1 Promoter Modulates Transcription. J. Am. Chem. Soc. 138, 2563-2570.
(38) Dai, J., Carver, M., and Yang, D. (2008) Polymorphism of human telomeric quadruplex structures. Biochimie 90, 1172-1183.
(39) Kim, M., Kreig, A., Lee, C. Y., Rube, H. T., Calvert, J., Song, J. S., and Myong, S. (2016) Quantitative analysis and prediction of G-quadruplex forming sequences in double-stranded DNA. Nucleic Acids Res. 44, 4807-4817.
(40) Cheng, M., Cheng, Y., Hao, J., Jia, G., Zhou, J., Mergny, J. L., and Li, C. (2018) Loop permutation affects the topology and stability of G-quadruplexes. Nucleic Acids Res. 46, 9264-9275.
(41) Chen, M. C., Tippana, R., Demeshkina, N. A., Murat, P., Balasubramanian, S., Myong, S., and Ferre-D'Amare, A. R. (2018) Structural basis of G-quadruplex unfolding by the DEAH/RHA helicase DHX36. Nature 558, 465-469.
(42) Barshai, M., and Orenstein, Y. (2019) Predicting G-Quadruplexes from DNA Sequences Using Multi-Kernel Convolutional Neural Networks, In Proceedings of the 10th ACM International Conference on Bioinformatics, Computational Biology and Health Informatics, pp 357-365, Association for Computing Machinery, Niagara Falls, N.Y., USA, DOI: 10.1145/3307339.3342133.
(43) Levens, D., Baranello, L., and Kouzine, F. (2016) Controlling gene expression by DNA mechanics: emerging insights and challenges. Biophys. Rev. 8, 23-32.
(44) Chao, W., and D'Amore, P. A. (2008) IGF2: epigenetic regulation and role in development and disease. Cytokine Growth Factor Rev. 19, 111-120.
(45) Chapple, C. E., Robisson, B., Spinelli, L., Guien, C., Becker, E., and Brun, C. (2015) Extreme multifunctional proteins identified from a human protein interaction network. Nat. Commun. 6, 7412.
(46) Phan, A. T., and Patel, D. J. (2003) Two-repeat human telomeric d(TAGGGTTAGGGT) (SEQ ID NO: 53) sequence forms interconverting parallel and antiparallel G-quadruplexes in solution: distinct topologies, thermodynamic properties, and folding/unfolding kinetics. J. Am. Chem. Soc. 125, 15021-15027.
(47) Rodriguez, R., Miller, K. M., Forment, J. V., Bradshaw, C. R., Nikan, M., Britton, S., Oelschlaegel, T., Xhemalce, B., Balasubramanian, S., and Jackson, S. P. (2012) Small-moleculeinduced DNA damage identifies alternative DNA structures in human genes. Nat. Chem. Biol. 8, 301-310.
(48) Wagih, O. (2017) ggseqlogo: a versatile R package for drawing sequence logos. Bioinformatics 33, 3645-3647.

### PART B

### Introduction

G-quadruplexes (G4s) are four-stranded secondary structures formed in guanine-rich nucleic acids [1]. The building block of G4s is the G-tetrad, consisting of four guanines connected through Hoogsteen hydrogen bonds in a cyclic coplanar arrangement [2]. A G4 structure is formed when two or more G-tetrad planes stack on top of each other and is stabilized by physiological relevant monovalent cations, especially K+ [3-5]. The biologically relevant intramolecular G4s are globular nucleic acid structures with unique folding and capping structures that provide an opportunity for selective targeting by small molecules [6-8].

G4 structures are involved in many cellular processes of DNA, including gene transcription [9,10], DNA replication [11], and genome stability [12,13]. In the human genome, G4 structures are prevalent in the regulatory regions and enriched in the promoters of cancer-related genes [14,15]. In particular, MYC, one of the most deregulated oncogenes in human cancer, has a DNA-G4 forming motif (MycG4) in its promoter [9,16-20]. Compounds that bind and stabilize the MycG4 structure have been shown to repress MYC expression and lead to cancer cell death [8,9,16]. Therefore, the MycG4 is considered an attractive target for anticancer drugs. However, over 10,000 G4 structures have been discovered in human chromatin of precancerous cells [15,21]. It is thus important to determine the selectivity of a G4-targeting compound.

3,6-Bis(1-methyl-4-vinylpyridinium) carbazole diiodide (BMVC) is a G4-interactive compound and the first fluorescent probe (λex,max=435, λem,max=580) to detect G4 structures in human cells [22-24]. BMVC has also been developed as a potential fluorescent marker for cancer cells [25,26]. Whereas BMVC was first developed to detect G4 structures in human telomeres, a recent study shows that BMVC binds the MYC promoter G4 (MycG4, FIG. 1b) with higher selectivity and affinity [27]. The solution structures of BMVC-MycG4 complexes have been determined, and show that BMVC binds to the MycG4 via multiple interactions, including stacking external G-tetrads, recognition of the MycG4-flanking bases, and conformational adjustment of the BMVC molecule. Moreover, the results show BMVC represses MYC expression in a human breast cancer cell line. However, the binding selectivity of BMVC to potential G4s formed in the human chromatin has not been broadly examined.

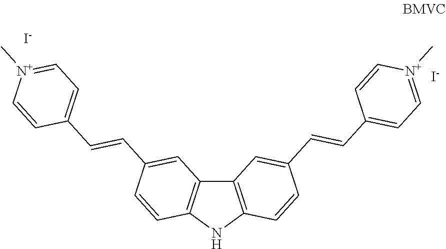

Microarray glass slides with hundred thousands of DNA sequences are a fast, straightforward, and high-throughput platform that has been employed to screen, profile, and quantify ligand and protein interactions with DNA and RNA molecules [28-30]. As described herein custom DNA microarrays have been designed that can assess the binding selectivities of proteins, small molecules, and antibodies across over 15,000 potential G4 structures [31].

Herein, is described a binding-selectivity analysis of BMVC to the MycG4 and other G4 structures using custom G4 microarrays and competition experiments between Cy5-fluorophore (λ_{ex,max}=647, λ_{em,max}=665) labeled small molecule pyridostatin [32] (Cy5-PDS) and unlabeled BMVC. The results show that BMVC differentially binds to various G4 structures and has a different G4 selectivity profile from Cy5-PDS. BMVC shows preferential binding to the MycG4 among the known G4 structures. Moreover, the microarray data reveals the sequence selectivity of BMVC to the flanking residues of the MycG4, especially at the 3′-end. The large-scale microarray results are confirmed by orthogonal small-scale NMR and fluorescence binding analyses. This is the first large-scale study of a G4-interactive ligand that shows a high-throughput evaluation of G4-binding selectivity and sequence specificity with unbiased selection of G4 sequences. It demonstrates the potential of custom DNA microarrays in the development of drugs targeting DNA or RNA structures.

### Results

BMVC Binds G4 Sequences Differently from PDS

Custom G4 microarrays have been designed that contain a total of 19,249 G4 DNA sequences [31, the entirety of disclosure of which, including the supplemental information, is incorporated herein by reference]. The G4 microarrays were created by covalently attaching thousands of unique G4-forming DNA 60-mers to a glass surface. Pyridostatin (PDS) is a known G4-interactive compound. Measured by the fluorescence intensity of Cy5-PDS bound to each sequence in potassium-containing solution, Cy5-PDS was shown to preferentially bind G4-forming sequences on the G4 microarrays [31]. To test the binding selectivity of BMVC, competition experiments using custom G4 microarrays were performed. The addition of potassium-containing solution to G4-forming oligonucleotides induced G4 formation. Subsequently, the microarrays were incubated with 1 μM Cy5-PDS in the absence or presence of 1 μM, 3 μM, or 10 μM of the unlabeled BMVC molecule. After washing to remove the unbound Cy5-PDS and BMVC, the fluorescence intensities of Cy5-PDS bound to DNA oligonucleotides were detected using a fluorescence scanner. The binding selectivity of BMVC to different G4 structures was assessed by measuring the relative fluorescence intensity reduction of Cy5-PDS as BMVC concentration increased.

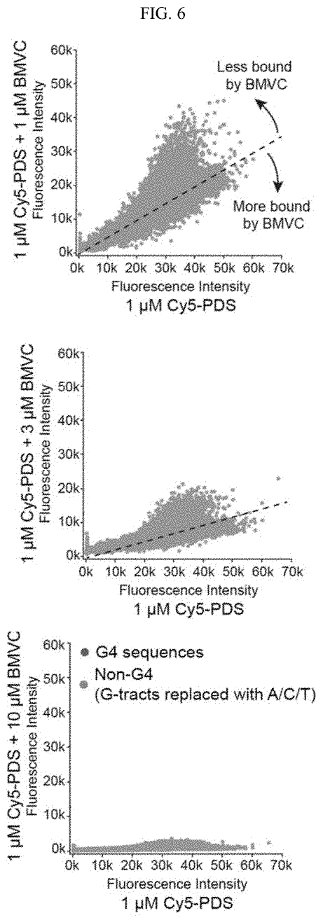

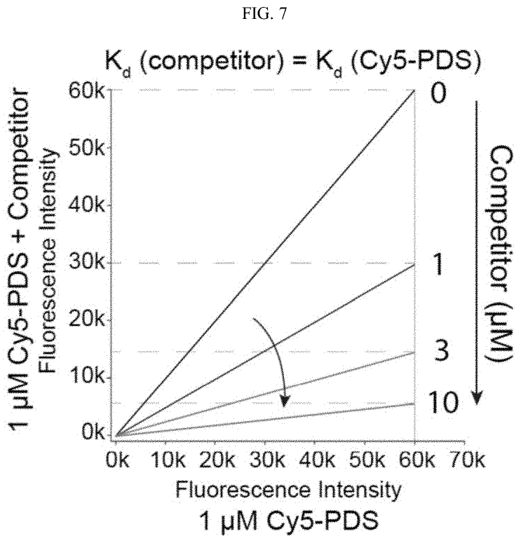

The fluorescence intensities of 1 μM Cy5-PDS in the presence of various concentrations of unlabeled BMVC were plotted against the fluorescence intensities in the absence of BMVC (FIG. 6). The competition experiment of 1 μM Cy5-PDS with 1 μM of unlabeled PDS was performed as the positive control. For a compound that competitively binds all sequences with the same affinity as Cy5-PDS, the competition experiments of 1 μM Cy5-PDS with various concentrations of the unlabeled compound will follow the predicted linear relationships (FIG. 7). Furthermore, fluorescence intensities of Cy5-PDS bound to various G4 sequences will uniformly decrease in a dose-dependent manner, as presented by decreased slopes (FIG. 7). In the competition experiment, the unlabeled BMVC could compete with the Cy5-PDS binding to G4 sequences in a dose-dependent manner (FIG. 6). However, the binding profile of BMVC was different from unlabeled PDS. Selectivity can be better assessed at equimolar concentrations of unlabeled ligand and Cy5-PDS (both 1 μM) (FIG. 6, top graph). BMVC displays a more pronounced binding selectivity to different G4 sequences, as shown by a larger deviation from linear relationships, particularly with the stable G4 forming sequences (at higher fluorescence intensities, FIG. 6). Unlabeled PDS appears to bind less selectively to the G4 sequences than BMVC, as shown by the stronger competition at the weaker Cy5-PDS-bound sequences (non-G4 sequences) (at lower fluorescence intensities).

### BMVC Shows Different Binding Selectivity to Various G4 Structures as Compared to PDS

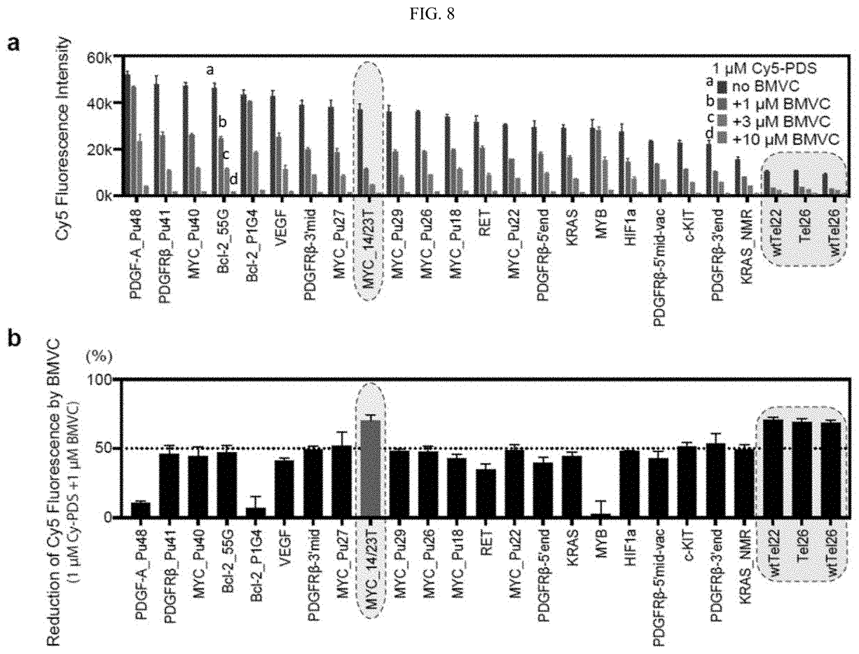

To determine the G4-binding selectivity of BMVC, the BMVC binding to known G4 structures was examined, including 7 well-studied MYC promoter G4 sequences, 15 other oncogene promoter G4 sequences, and 3 human telomeric G4 sequences (TABLE 4). BMVC competes with the binding of Cy5-PDS to most G4 sequences in a dose-dependent manner as indicated by reduced fluorescence intensities (FIG. 8a).

TABLE 4
G4 Sequences Analyzed In FIG. 8.

| Name [reference] | G4 Sequence (5′→3′) | SEQ ID NO: |
|---|---|---|
| MYC_Pu40 [9] | TTATGGGGAGGGTGGGGAGGGTGGGGAAGGTGGGGAGGAG | 42 |
| MYC_Pu29 [9] | TTGGGGAGGGTGGGGAGGGTGGGGAAGGT | 43 |
| MYC_Pu27 [9] | TGGGGAGGGTGGGGAGGGTGGGGAAGG | 10 |
| MYC_Pu26 [33, 34] | TTGGGGAGGGTGGGGAGGGTGGGGAA | 9 |
| MYC_Pu22 [35, 36] | TGAGGGTGGGGAGGGTGGGGAA | 6 |
| MYC_14/23T [35, 36] | TGAGGGTGGGTAGGGTGGGTAA | 4 |
| MYC_Pu18 [37] | AGGGTGGGGAGGGTGGGG | 7 |
| PDGFRβ_Pu41 [38] | GCTGGGAGAAGGGGGGGCGGCGGGGCAGGGAGGGTGGACGC | 44 |
| PDGFRβ-5′end [38] | TTGGGAGAAGGGGGGGCGGCGGGGCA | 45 |
| PDGFRβ-5′mid-vac [39] | AAGGGAGGGCGGCGGGGCA | 46 |
| PDGFRβ-3′mid [40] | AAGGGGGGGCGGCGGGGCAGGGAGGGT | 47 |
| PDGFRβ-3′end [41] | CGGCGGGGCAGGGAGGGTGGACG | 26 |
| wtTel22 [42] | AGGGTTAGGGTTAGGGTTAGGG | 48 |
| Tel26 [43-45] | TTAGGGTTAGGGTTAGGGTTAGGGAAA | 49 |
| wtTel26 [45, 46] | TTAGGGTTAGGGTTAGGGTTAGGGTTA | 50 |
| Bcl-2_55G [47] | AGGGGCGGGCGCGGGAGGAAGGGGGCGGGA | 17 |
| Bcl-2 P1G4 [48] | CGGGCGGGAGCGCGGCGGGCGGGCGGGC | 18 |
| PDGF-A_Pu48 [49] | GGAGGCGGGGGGGGGGGGGCGGGGGCGGGGGCGGGGGAGG | 24 |
| | GGCGCGGC | |
| KRAS [50] | AGGGCGGTGTGGGAAGAGGGAAGAGGGGGAGGCAG | 12 |
| KRAS_NMR [51] | AGGGCGGTGTGGGAATAGGGAA | 13 |
| VEGF [52] | CGGGGCGGGCCGGGGGCGGGGT | 15 |
| RET [53] | GGGTAGGGGCGGGGCGGGGCGGGGGC | 23 |
| MYB [54] | GGAGGAGGAGGTCACGGAGGAGGAGGAGAAGGAGGAGGAG | 20 |
| | GA | |
| HIF1a [55] | GGGAGGGAGAGGGGGCGGG | 19 |
| c-KIT [56] | AGGGAGGGCGCTGGGAGGAGGG | 16 |

Comparison of the inhibitory effects for the known G4 structures revealed differential G4 binding selectivity of BMVC vs. Cy5-PDS (FIG. 8b). G4 sequences were ranked based on the fluorescence intensity of bound Cy5-PDS. As illustrated in FIG. 8a bars labeled a), Cy5-PDS prefers long and highly G-rich sequences, such as PDGF-A_Pu48, PDGFRb_Pu41, and MYC Pu40. In addition, it also binds well to parallel G4s, such as Bcl-2_55G, Bcl-2_P1G4, VEFG, and various MYC G4s. For most G4s, the fluorescence intensity of 1 μM Cy5-PDS was reduced by 50% upon equimolar addition of BMVC (FIG. 8b), suggesting a similar binding affinity of BMVC and Cy5-PDS to these G4s. However, the binding of BMVC was much weaker for the PDGF-A_Pu48 and MYB sequences than the binding of Cy5-PDS (FIG. 8b). Both PDGF-A_Pu48 and MYB sequences have no-5′-flanking, while MYB forms a tetrad-heptad structure [54], whereas the optimal binding of BMVC requires a flanking base at both the 5′-end and 3′-end, as shown by NMR solution structural study of the BMVC-MycG4 complex [27].

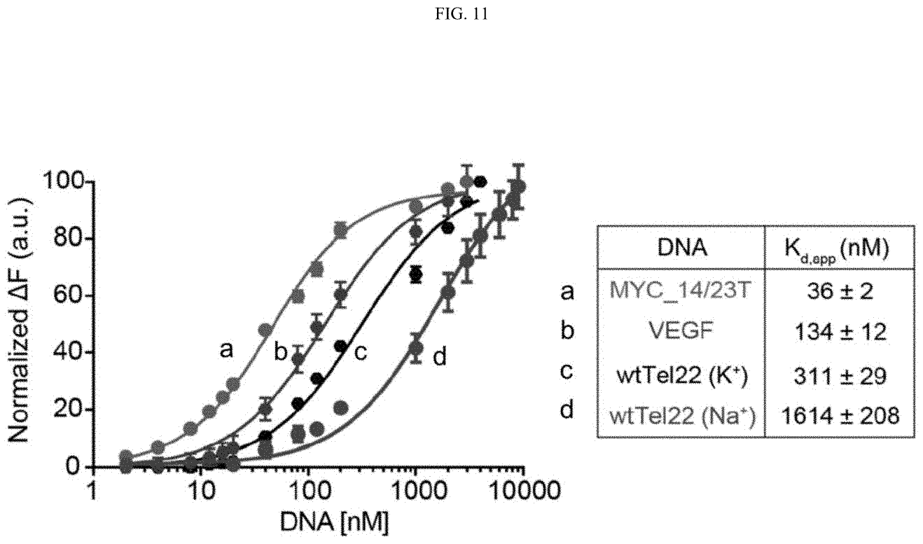

Cy5-PDS binds appears to bind less well to nonparallel G4s, such as human telomeric G4s, which show less than 25% fluorescence intensity as compared to parallel-stranded G4s (FIG. 8a). BMVC significantly inhibited the binding of Cy5-PDS to human telomeric G4s (FIG. 8b), indicating a stronger binding of BMVC to the human telomeric G4s as compared to PDS. However, fluorescence measurements showed that BMVC binds parallel G4s, such as MYC and VEGF G4s, stronger than the human telomeric G4s (FIG. 11). Therefore, the microarray competition result indicates that Cy5-PDS binds the human telomeric G4s even weaker than BMVC.

### BMVC Preferentially Binds to MYC_14/23T Among the Known G4 Structures

In general, Cy5-PDS and BMVC both strongly bind to parallel G4s (FIG. 8b). Intriguingly, among all parallel G4s, BMVC induced largest reduction of the Cy5-PDS binding to the MYC_14/23T G4. It is important to note that Cy5-PDS also binds the MYC_14/23T G4 sequence very well (FIG. 8a), therefore the strongest competition effect demonstrates that BMVC selectively recognizes the MYC_14/23T G4. The MYC promoter G4 is the best-studied promoter G-quadruplex structure and a prototype of parallel G4s [8]. Notably, MYC_14/23T and MYC_Pu22 form the same parallel G4 (FIG. 8b) except for the 3′-end flanking residue, which is a T in MYC_14/23T and a G in MYC_Pu22 [35]. The strikingly stronger binding of BMVC to MYC_14/23T than MYC_Pu22 (FIG. 8b) indicates that BMVC selectively recognizes the 3′-flanking T of MYC_14/23T G4.

### 2.4. BMVC Selectively Recognizes the Flanking Sequences of Parallel

### G4S, Especially the 3′-Flanking T

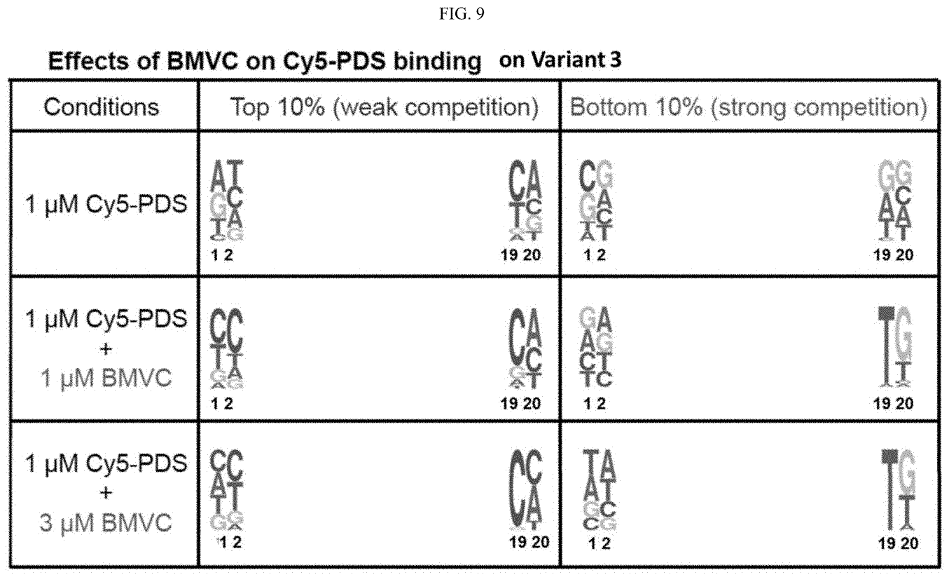

To examine the preference of BMVC for specific flanking sequences, the binding of BMVC to MYC G4-derived sequence variants of the two flanking bases at both ends (5′-NNGGGTGGGGAGGGTGGGNN-3′ (SEQ ID NO: 3), variant 3) was examined using the competition microarray experiments. The differential reduction of Cy5-PDS binding to variants in the flanking sequences induced by BMVC addition reveals the binding selectivity for specific MYC G4 flanking sequences. In the absence of BMVC, Cy5-PDS exhibits a slight preference for the 3′-flanking C and T, as shown by the most-bound (top 10%) and least-bound (bottom 10%) flanking variants (FIG. 9, top panel). The addition of BMVC significantly altered the most and least Cy5-PDS-bound flanking variants, with a clearly stronger selectivity at the 3′-end than at the 5′-end (FIG. 9, middle and bottom panels). The most and least Cy5-PDS-bound flanking variants in the presence of equimolar BMVC reveal the binding selectivity of BMVC. Particularly, thymine became markedly less enriched in the top 10% Cy5-PDS most-bound 3′-flanking variants but significantly enriched in the bottom 10% Cy5-PDS least-bound variants, indicating that BMVC strongly prefers the MYC G4 with the 3′-flanking T. On the other hand, C is the least-favored flanking base for BMVC binding at both the 3′- and 5′-ends, as shown by the greater enrichment in the top 10% Cy5-PDS most-bound flanking variants.

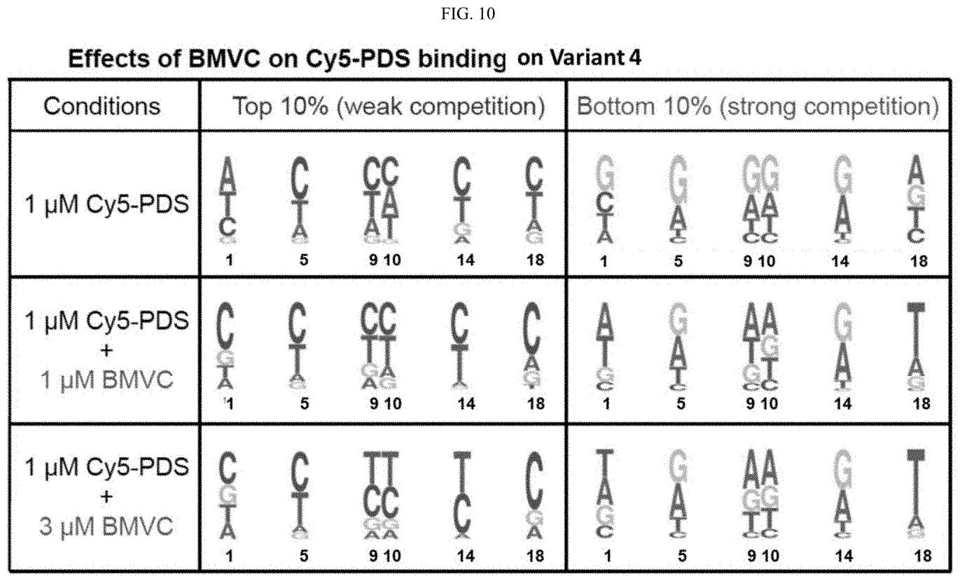

The effects of BMVC on the Cy5-PDS-binding to MYC G4 loop and single flanking-base sequence variants (5′-NGGGNGGGNNGGGNGGGN-3′ (SEQ ID NO: 2), variant 4) which include all possible loop and flanking variants (FIG. 10) was analyzed. Consistent with the two-base-flanking variants, the results showed BMVC strongly preferred the 3′-end flanking T but disfavored the flanking C at both ends. In contrast, Cy5-PDS preferred C for all three loops and the 3′-end flanking. It is noted that the MYC G4 single flanking-base variants all contain additional 3′-flanking bases for linking the G4 oligos to the microarray plates.

The sequence selectivity shown by the flanking variants explains the markedly weaker binding of BMVC to Bcl-2_P1G4 (FIG. 8). Bcl2_55G and Bcl-2_P1G4 both form parallel G4s with a long central loop (13-nucleotide (nt) long in Bcl2_55G and 12-nt long in Bcl-2_P1G4) but different flanking sequences [47,48]. However, whereas BMVC showed good binding to Bcl2_55G similar to other parallel G4s, the binding to Bcl-2_P1G4 was markedly weaker (FIG. 8 a,b). Bcl-2_P1G4 has a flanking C at both the 5′- and 3′-ends and only contains a short 1-nt flanking at the 5′-end, suggesting that BMVC disfavors the flanking C and short flanking.

### NMR Binding Experiments Confirm the Binding Selectivity of BMVC to G4 Structures and Flanking Sequences

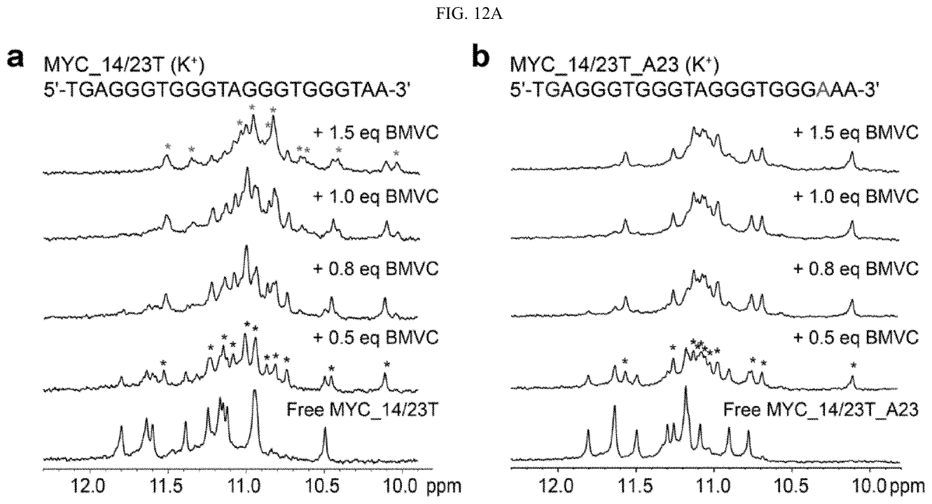

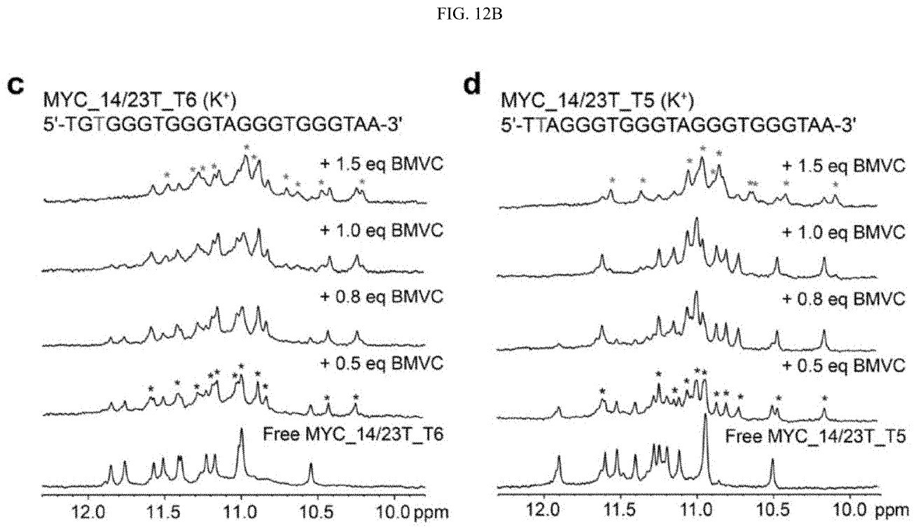

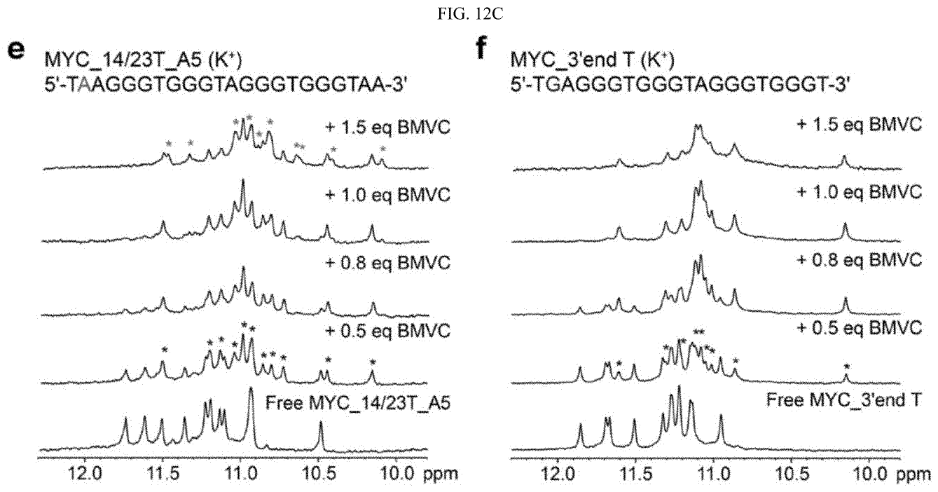

The binding selectivity of BMVC to G4 structures and flanking sequences was confirmed by NMR titration experiments of BMVC to different G4 sequences, including parallel-stranded MYC_14/23T G4 and its 5′- and 3′-flanking variants, VEGF and MYC1234 G4s, basket-type human telomeric G4 (wtTel22 in Na+), and hybrid type human telomeric G4 (Tel26 in K+) (FIG. 12). BMVC binds best to the MYC_14/23T G4, as indicated by well-resolved imino proton peaks for BMVC complexes (FIG. 12, panel a). A previous NMR solution structural study shows that BMVC binds at both ends of the MYC_14/23T G4 to form a 2:1 complex [27]. Mutations at the 5′-flanking sequence do not affect the binding of BMVC at the 5′-end. In contrast, the 3′-end binding of BMVC is sensitive to the mutations at the 3′-flanking sequence, with a clear preference for the 3′-flanking T. In addition, BMVC prefers at least two flanking bases for a specific binding. These results are in good agreement with the DNA microarray data (FIGS. 9 and 10).

While BMVC binds the MYC_14/23T G4 with the highest affinity (FIG. 11), BMVC can bind well to other parallel G4s, such as MYC1234 and VEGF G4 (FIG. 12, panels b and d). Additionally, BMVC favors the 5′-flanking A of parallel G4s, as indicated in the NMR titration data of the VEGF G4 flanking variants (FIG. 12, panels c and d). However, BMVC did not show specific binding to the basket-type or hybrid-type human telomeric G4s (see FIG. 12, panels e and f). These results are consistent with the G4 microarray data.

### Conclusions

A high-throughput, large-scale custom G4 DNA microarray to assess the binding selectivities of proteins and small molecules across 20,000 potential G4 structures simultaneously has been established. Competition binding experiments of the Cy5 labeled PDS and the unlabeled G4-interactive small molecule BMVC demonstrate that the custom G4 microarray platform can assess the binding selectivity of BMVC to various G4 structures and flanking sequences, as well as differential G4 binding selectivity between BMVC and PDS. The results reveal that BMVC selectively binds parallel G4s, in particular the MYC_14/23T G4. Moreover, the G4 microarray data shows BMVC selectively recognizes the flanking sequences of parallel G4s, especially the 3′-flanking T. Importantly, the binding and sequence selectivity revealed by the large-scale DNA microarray data is in good agreement with the individual binding data by NMR and fluorescence. It has been found that the G4 DNA microarray provides a high-throughput and unbiased platform to assess the binding selectivity of G4-targeting molecules on a large scale and can help understand the properties that govern molecular recognition.

### Materials and Methods

### Custom G4 DNA Microarray Design

A custom microarray was designed that contains four identical sectors that contain ca.177,440 ssDNA 60-mers to examine G4 binding selectivity (NCBI GEO Platform GPL28372). The microarray contains different sets of G4 variants designed to examine several sequence parameters that affect G4 formation and binding selectivity such as loop length, loop sequence, flanking tail sequence, and single nucleotide variants of known G4s [31]. Briefly, the array includes a set of sequences from human telomeres and oncogene promoters known to form G4s with various topologies as positive controls (TABLE 4) as well as a set of 295 additional G4-forming sequences from the literature [57]. Loop and flanking tail sequences were varied using A, T, G, and C polynucleotide stretches and a subset of combinations, described in [31]. For the flanking variants, 256 versions of the major MYC G4 with all possible dinucleotide flanking sequences (5′-NNGGGTGGGGAGGGTGGGNN-3′ (SEQ ID NO: 3)) were generated. For the loop sequence variants, 4,096 sequences of the form 5′-NGGGNGGGNNGGGNGGGN-3′ (SEQ ID NO: 2) were generated. Negative controls include 19 oncogene G4s in which all G-tracts are replaced with either A, T, or C, reverse complements of G4 sequences, as well as a set of 86 published non-G4 sequences [57].

DNA Microarray Binding Experiments DNA microarray experiments were performed and analyzed as described previously [31]. Microarrays were preincubated with a pH 7.4 phosphate buffer solution with 100 mM potassium for 1 h at room temperature to induce G4 formation. Arrays then were blocked with 4% nonfat dry-milk in a potassium phosphate buffer before incubation with small molecules (Cy5-PDS, Cy5-PDS+BMVC, or Cy5-PDS+PDS) for 1 h at room temperature.

Data Processing and Analysis Molecule-bound microarrays were scanned with an Agilent G5761A SureScan Dx Microarray Scanner System to detect Cy5 signal at two laser settings (30 and 100 PMT). Spot intensities from microarray images were extracted using Agilent Feature Extraction Software and are reported as raw fluorescence intensities. All binding assays were performed twice with high agreement between replicates (R>0.8). Microarrays with the fewest number of saturated spots were used for further analysis. Median intensity was then computed for probes containing identical sequence on each microarray. Sequence logos were generated from a position frequency matrix generated from selected sequences using ggseqlogo [58].

### NMR Spectroscopy Experiments

G4 DNA oligonucleotides were synthesized using β-cyanoethylphosphoramidite solid-phase chemistry (Applied Biosystem Expedite 8909), as described previously [36]. NMR experiments were performed on a Bruker AV-III-500-HD equipped with a BBFO Z-gradient cryoprobe. DNA samples were heated to 95° C. for 5 min, then cooled slowly for G4 formation. For the 1D 1H NMR experiments, samples contained 100-250 μM DNA in an appropriate buffer solution with 10% D2O for the lock. The titrations were performed by adding increasing amounts of the compounds to the DNA samples in solution.

### REFERENCES FOR PART B (ENCLOSED IN BRACKETS)

1. Yang, D. G-Quadruplex DNA and RNA. Methods Mol. Biol. 2019, 2035, 1-24.
2. Gellert, M.; Lipsett, M. N.; Davies, D. R. Helix formation by guanylic acid. Proc. Natl. Acad. Sci. USA 1962, 48, 2013-2018.
3. Williamson, J. R.; Raghuraman, M. K.; Cech, T. R. Monovalent cation-induced structure of telomeric DNA: The G-quartet model. Cell 1989, 59, 871-880.
4. Sen, D.; Gilbert, W. A sodium-potassium switch in the formation of four-stranded G4-DNA. Nature 1990, 344, 410-414.
5. Hud, N. V.; Smith, F. W.; Anet, F. A. L.; Feigon, J. The selectivity for K+ versus Na+ in DNA quadruplexes is dominated by relative free energies of hydration: A thermodynamic analysis by 1H NMR. Biochemistry 1996, 35, 15383-15390.
6. Neidle, S. Quadruplex nucleic acids as novel therapeutic targets. J. Med. Chem. 2016, 59, 5987-6011.
7. Yang, D.; Okamoto, K. Structural insights into G-quadruplexes: Towards new anticancer drugs. Future Med. Chem. 2010, 2, 619-646.
8. Chen, Y.; Yang, D. Sequence, stability, and structure of G-quadruplexes and their interactions with drugs. Curr. Protoc. Nucleic Acid Chem. 2012, 50, 17.5.1-17.5.17.
9. Siddiqui-Jain, A.; Grand, C. L.; Bearss, D. J.; Hurley, L. H. Direct evidence for a G-quadruplex in a promoter region and its targeting with a small molecule to repress c-MYC transcription. Proc. Natl. Acad. Sci. USA 2002, 99, 11593-11598.
10. Gray, L. T.; Vallur, A. C.; Eddy, J.; Maizels, N. G quadruplexes are genomewide targets of transcriptional helicases XPB and XPD. Nat. Chem. Biol. 2014, 10, 313-318.
11. Bochman, M. L.; Paeschke, K.; Zakian, V. A. DNA secondary structures: Stability and function of G-quadruplex structures. Nat. Rev. Genet. 2012, 13, 770-780.
12. Piazza, A.; Boule, J. B.; Lopes, J.; Mingo, K.; Largy, E.; Teulade-Fichou, M. P.; Nicolas, A. Genetic instability triggered by G-quadruplex interacting Phen-DC compounds in Saccharomyces cerevisiae. Nucleic Acids Res. 2010, 38, 4337-4348.
13. Ribeyre, C.; Lopes, J.; Boule, J. B.; Piazza, A.; Guedin, A.; Zakian, V. A.; Mergny, J. L.; Nicolas, A. The yeast Pif1 helicase prevents genomic instability caused by G-quadruplex-forming CEB1 sequences in vivo. PLoS Genet 2009, 5, e1000475.
14. Huppert, J. L.; Balasubramanian, S. G-quadruplexes in promoters throughout the human genome. Nucleic Acids Res. 2007, 35, 406-413.
15. Hansel-Hertsch, R.; Beraldi, D.; Lensing, S. V.; Marsico, G.; Zyner, K.; Parry, A.; Di Antonio, M.; Pike, J.; Kimura, H.; Narita, M.; et al. G-quadruplex structures mark human regulatory chromatin. Nat. Genet. 2016, 48, 1267-1272.
16. Brooks, T. A.; Hurley, L. H. The role of supercoiling in transcriptional control of MYC and its importance in molecular therapeutics. Nat. Rev. Cancer 2009, 9, 849-861.
17. Simonsson, T.; Pecinka, P.; Kubista, M. DNA tetraplex formation in the control region of c-myc. Nucleic Acids Res. 1998, 26, 1167-1172.
18. DesJardins, E.; Hay, N. Repeated CT elements bound by zinc finger proteins control the absolute and relative activities of the two principal human c-myc promoters. Mol. Cell. Biol. 1993, 13, 5710-5724.
19. Michelotti, E. F.; Tomonaga, T.; Krutzsch, H.; Levens, D. Cellular nucleic acid binding protein regulates the CT element of the human c-myc protooncogene. J. Biol. Chem. 1995, 270, 9494-9499.
20. Wu, G.; Xing, Z.; Tran, E. J.; Yang, D. DDX5 helicase resolves G-quadruplex and is involved in MYC gene transcriptional activation. Proc. Natl. Acad. Sci. USA 2019, 116, 20453-20461.
21. Kouzine, F.; Wojtowicz, D.; Baranello, L.; Yamane, A.; Nelson, S.; Resch, W.; Kieffer-Kwon, K. R.; Benham, C. J.; Casellas, R.; Przytycka, T. M.; et al. Permanganate/S1 nuclease footprinting reveals non-B DNA structures with regulatory potential across a mammalian genome. Cell Syst. 2017, 4, 344-356.
22. Chang, C. C.; Wu, J. Y.; Chien, C. W.; Wu, W. S.; Liu, H.; Kang, C. C.; Yu, L. J.; Chang, T. C. A fluorescent carbazole derivative: High sensitivity for quadruplex DNA. Anal. Chem. 2003, 75, 6177-6183.
23. Chang, C. C.; Kuo, I. C.; Ling, I. F.; Chen, C. T.; Chen, H. C.; Lou, P. J.; Lin, J. J.; Chang, T. C. Detection of quadruplex DNA structures in human telomeres by a fluorescent carbazole derivative. Anal. Chem. 2004, 76, 4490-4494.
24. Chang, C. C.; Chu, J. F.; Kao, F. J.; Chiu, Y. C.; Lou, P. J.; Chen, H. C.; Chang, T. C. Verification of antiparallel G-quadruplex structure in human telomeres by using two-photon excitation fluorescence lifetime imaging microscopy of the 3,6-bis(1-methyl-4-vinylpyridinium)carbazole diiodide molecule. Anal. Chem. 2006, 78, 2810-2815.
25. Kang, C. C.; Chang, C. C.; Cheng, J. Y.; Chang, T. C. Simple method in diagnosing cancer cells by a novel fluorescence probe BMVC. J. Chin. Chem. Soc. 2005, 52, 1069-1072.
26. Chang, C. C.; Kuo, I. C.; Lin, J. J.; Lu, Y. C.; Chen, C. T.; Back, H. T.; Lou, P. J.; Chang, T. C. A novel carbazole derivative, BMVC: A potential antitumor agent and fluorescence marker of cancer cells. Chem. Biodivers. 2004, 1, 1377-1384.
27. Liu, W.; Lin, C.; Wu, G.; Dai, J.; Chang, T. C.; Yang, D. Structures of 1:1 and 2:1 complexes of BMVC and MYC promoter G-quadruplex reveal a mechanism of ligand conformation adjustment for G4-recognition. Nucleic Acids Res. 2019, 47, 11931-11942.
28. Berger, M. F.; Bulyk, M. L. Universal protein-binding microarrays for the comprehensive characterization of the DNA-binding specificities of transcription factors. Nat. Protoc. 2009, 4, 393-411.
29. Badis, G.; Berger, M. F.; Philippakis, A. A.; Talukder, S.; Gehrke, A. R.; Jaeger, S. A.; Chan, E. T.; Metzler, G.; Vedenko, A.; Chen, X.; et al. Diversity and complexity in DNA recognition by transcription factors. Science 2009, 324, 1720-1723.
30. Iida, K.; Nakamura, T.; Yoshida, W.; Tera, M.; Nakabayashi, K.; Hata, K.; Ikebukuro, K.; Nagasawa, K. Fluorescent-ligand-mediated screening of G-quadruplex structures using a DNA microarray. Angew. Chem. Int. Ed. 2013, 52, 12052-12055.
31. Ray, S.; Tillo, D.; Boer, R. E.; Assad, N.; Barshai, M.; Wu, G.; Orenstein, Y.; Yang, D.; Schneekloth, J. S., Jr.; Vinson, C. Custom DNA microarrays reveal diverse binding preferences of proteins and small molecules to thousands of G-quadruplexes. ACS Chem. Biol. 2020, 15, 925-935.
32. Muller, S.; Kumari, S.; Rodriguez, R.; Balasubramanian, S. Small-molecule-mediated G-quadruplex isolation from human cells. Nat. Chem. 2010, 2, 1095-1098.
33. Phan, A. T.; Modi, Y. S.; Patel, D. J. Propeller-type parallel-stranded G-quadruplexes in the human c-myc promoter. J. Am. Chem. Soc. 2004, 126, 8710-8716.
34. Dickerhoff, J.; Onel, B.; Chen, L.; Chen, Y.; Yang, D. Solution structure of a MYC promoter G-quadruplex with 1:6:1 loop length. ACS Omega 2019, 4, 2533-2539.
35. Ambrus, A.; Chen, D.; Dai, J.; Jones, R. A.; Yang, D. Solution structure of the biologically relevant G-quadruplex element in the human c-MYC promoter. Implications for G-quadruplex stabilization. Biochemistry 2005, 44, 2048-2058.
36. Dai, J.; Carver, M.; Hurley, L. H.; Yang, D. Solution structure of a 2:1 quindoline-c-MYC G-quadruplex: Insights into G-quadruplex-interactive small molecule drug design. J. Am. Chem. Soc. 2011, 133, 17673-17680.
37. Seenisamy, J.; Rezler, E. M.; Powell, T. J.; Tye, D.; Gokhale, V.; Joshi, C. S.; Siddiqui-Jain, A.; Hurley, L. H. The dynamic character of the G-quadruplex element in the c-MYC promoter and modification by TMPyP4. J. Am. Chem. Soc. 2004, 126, 8702-8709.
38. Qin, Y.; Fortin, J. S.; Tye, D.; Gleason-Guzman, M.; Brooks, T. A.; Hurley, L. H. Molecular cloning of the human platelet-derived growth factor receptor beta (PDGFR-beta) promoter and drug targeting of the G-quadruplex-forming region to repress PDGFR-beta expression. Biochemistry 2010, 49, 4208-4219.
39. Wang, K. B.; Dickerhoff, J.; Wu, G.; Yang, D. PDGFR-beta Promoter Forms a Vacancy G-Quadruplex that Can Be Filled in by dGMP: Solution Structure and Molecular Recognition of Guanine Metabolites and Drugs. J. Am. Chem. Soc. 2020, 142, 5204-5211.
40. Chen, Y.; Agrawal, P.; Brown, R. V.; Hatzakis, E.; Hurley, L. H.; Yang, D. The major G-quadruplex formed in the human platelet-derived growth factor receptor beta promoter adopts a novel broken-strand structure in K+ solution. J. Am. Chem. Soc. 2012, 134, 13220-13223.
41. Onel, B.; Carver, M.; Agrawal, P.; Hurley, L. H.; Yang, D. The 3′-end region of the human PDGFR-beta core promoter nuclease hypersensitive element forms a mixture of two unique end-insertion G-quadruplexes. Biochim. Biophys. Acta Gen. Subj. 2018, 1862, 846-854.
42. Wang, Y.; Patel, D. J. Solution structure of the human telomeric repeat d[AG3(T2AG3)3] (SEQ ID NO: 48) G-tetraplex. Structure 1993, 1, 263-282.
43. Ambrus, A.; Chen, D.; Dai, J.; Bialis, T.; Jones, R. A.; Yang, D. Human telomeric sequence forms a hybrid-type intramolecular G-quadruplex structure with mixed parallel/antiparallel strands in potassium solution. Nucleic Acids Res. 2006, 34, 2723-2735.
44. Luu, K. N.; Phan, A. T.; Kuryavyi, V.; Lacroix, L.; Patel, D. J. Structure of the human telomere in K+ solution: An intramolecular (3+1) G-quadruplex sca old. J. Am. Chem. Soc. 2006, 128, 9963-9970.
45. Dai, J.; Punchihewa, C.; Ambrus, A.; Chen, D.; Jones, R. A.; Yang, D. Structure of the intramolecular human telomeric G-quadruplex in potassium solution: A novel adenine triple formation. Nucleic Acids Res. 2007, 35, 2440-2450.
46. Phan, A. T.; Luu, K. N.; Patel, D. J. Di erent loop arrangements of intramolecular human telomeric (3+1) G-quadruplexes in K+ solution. Nucleic Acids Res. 2006, 34, 5715-5719.
47. Agrawal, P.; Lin, C.; Mathad, R. I.; Carver, M.; Yang, D. The major G-quadruplex formed in the human BCL-2 proximal promoter adopts a parallel structure with a 13-nt loop in K+ solution. J. Am. Chem. Soc. 2014, 136, 1750-1753.
48. Onel, B.; Carver, M.; Wu, G.; Timonina, D.; Kalarn, S.; Larriva, M.; Yang, D. A New G-quadruplex with hairpin loop immediately upstream of the human BCL2 P1 promoter modulates transcription. J. Am. Chem. Soc. 2016, 138, 2563-2570.
49. Qin, Y.; Rezler, E. M.; Gokhale, V.; Sun, D.; Hurley, L. H. Characterization of the G-quadruplexes in the duplex nuclease hypersensitive element of the PDGF-A promoter and modulation of PDGF-A promoter activity by TMPyP4. Nucleic Acids Res. 2007, 35, 7698-7713.
50. Morgan, R. K.; Batra, H.; Gaerig, V. C.; Hockings, J.; Brooks, T. A. Identification and characterization of a new G-quadruplex forming region within the kRAS promoter as a transcriptional regulator. Biochim. Biophys. Acta 2016, 1859, 235-245.
51. Kerkour, A.; Marquevielle, J.; Ivashchenko, S.; Yatsunyk, L. A.; Mergny, J. L.; Salgado, G. F. High-resolution three-dimensional NMR structure of the KRAS proto-oncogene promoter reveals key features of a G-quadruplex involved in transcriptional regulation. J. Biol. Chem. 2017, 292, 8082-8091.
52. Agrawal, P.; Hatzakis, E.; Guo, K.; Carver, M.; Yang, D. Solution structure of the major G-quadruplex formed in the human VEGF promoter in K+: Insights into loop interactions of the parallel G-quadruplexes. Nucleic Acids Res. 2013, 41, 10584-10592.
53. Tong, X.; Lan, W.; Zhang, X.; Wu, H.; Liu, M.; Cao, C. Solution structure of all parallel G-quadruplex formed by the oncogene RET promoter sequence. Nucleic Acids Res. 2011, 39, 6753-6763.
54. Palumbo, S. L.; Memmott, R. M.; Uribe, D. J.; Krotova-Khan, Y.; Hurley, L. H.; Ebbinghaus, S. W. A novel G-quadruplex-forming GGA repeat region in the c-myb promoter is a critical regulator of promoter activity. Nucleic Acids Res. 2008, 36, 1755-1769.
55. De Armond, R.; Wood, S.; Sun, D.; Hurley, L. H.; Ebbinghaus, S. W. Evidence for the presence of a guanine quadruplex forming region within a polypurine tract of the hypoxia inducible factor 1alpha promoter. Biochemistry 2005, 44, 16341-16350.
56. Wei, D.; Husby, J.; Neidle, S. Flexibility and structural conservation in a c-KIT G-quadruplex. Nucleic Acids Res. 2015, 43, 629-644.
57. Bedrat, A.; Lacroix, L.; Mergny, J. L. Re-evaluation of G-quadruplex propensity with G4Hunter. Nucleic Acids Res. 2016, 44, 1746-1759.
58. Wagih, O. ggseqlogo: A versatile R package for drawing sequence logos. Bioinformatics 2017, 33, 3645-3647.

What is claimed is:

1. A method for determining binding preferences of a non-fluorescent test compound for one or more target G-quadruplex moieties, the method comprising;
a) incubating a device comprising a plurality of single-stranded nucleic acid molecules capable of forming one or more G-quadruplex moieties including the target G-quadruplex moieties with a solution comprising a G-quadruplex stabilizing cation selected from the group consisting of Na⁺ and K⁺;
b) incubating the device with a solution of a compound capable of providing a fluorescent signal (a fluorescent compound), wherein the fluorescent compound is capable of binding to the target G-quadruplex moieties;
c) measuring a first fluorescent signal from the fluorescent compound bound to the device;
d) removing the fluorescent compound from the device;
e) contacting the device with a solution of the fluorescent compound and the test compound;
f) measuring a second fluorescent signal from the fluorescent compound bound to the device; and
g) using the first fluorescent signal and the second fluorescent signal to calculate the binding preferences of the test compound.

2. The method of claim 1 wherein the device is a microarray comprising a plurality of single-stranded DNA molecules (s-DNAs) attached to a solid substrate; where
each s-DNA is from 50 nucleotides (nt) to 100 nt in length and
includes an independently selected linker sequence and an independently selected G-quadruplex-forming region (G4 sequence) where the G4 sequence has formula I
S1-T1-S2-T2-S3-T3-S4-T4-S5 (I) (SEQ ID NO: 54)
wherein T1 is G-Gx1, T2 is G-Gx2, T3 is G-Gx3, and T4 is G-Gx4;
S1 to S5 are independently selected sequences of from 0 to 5 nucleotides independently selected in each instance from the group consisting of A, T, C, and G; and
x1 to x4 are each independently selected in each instance from the group consisting of 2, 3, 4, and 5.

3. The method of claim 2 wherein the G-quadruplex stabilizing cation is K⁺.

4. The method of claim 2 wherein the G4 sequence is selected from the group consisting of
(SEQ ID NO: 42)
5′-TTATGGGGAGGGTGGGGAGGGTGGGGAAGGTGGGGAGGAG-3′,
(SEQ ID NO: 43)
5′-TTGGGGAGGGTGGGGAGGGTGGGGAAGGT-3′,
(SEQ ID NO: 10)
5′-TGGGGAGGGTGGGGAGGGTGGGGAAGG-3′,
(SEQ ID NO: 9)
5′-TTGGGGAGGGTGGGGAGGGTGGGGAA-3′,
(SEQ ID NO: 6)
5′-TGAGGGTGGGGAGGGTGGGGAA-3′,
(SEQ ID NO: 4)
5′-TGAGGGTGGGTAGGGTGGGTAA-3′,
(SEQ ID NO: 7)
5′-AGGGTGGGGAGGGTGGGG-3′,
(SEQ ID NO: 44)
5′-GCTGGGAGAAGGGGGGGCGGCGGGGCAGGGAGGGTGGACGC-3′,
(SEQ ID NO: 45)
5′-TTGGGAGAAGGGGGGGCGGCGGGGCA-3′,
(SEQ ID NO: 46)
5′-AAGGGAGGGCGGCGGGGCA-3′,
(SEQ ID NO: 47)
5′-AAGGGGGGGCGGCGGGGCAGGGAGGGT-3′,
(SEQ ID NO: 26)
5′-CGGCGGGGCAGGGAGGGTGGACG-3′,
(SEQ ID NO: 48)
5′-AGGGTTAGGGTTAGGGTTAGGG-3′,
(SEQ ID NO: 49)
5′-TTAGGGTTAGGGTTAGGGTTAGGGAAA-3′,
(SEQ ID NO: 50)
5′-TTAGGGTTAGGGTTAGGGTTAGGGTTA-3′,
(SEQ ID NO: 17)
5′-AGGGGCGGGCGCGGGAGGAAGGGGGCGGGA-3′,
(SEQ ID NO: 18)
5′-CGGGCGGGAGCGCGGCGGGCGGGCGGGC-3′,
(SEQ ID NO: 24)
5′-GGAGGCGGGGGGGGGGGGGCGGGGGCGGGGGCGGGGGAGGGGCG
CGGC-3′,
(SEQ ID NO: 12)
5′-AGGGCGGTGTGGGAAGAGGGAAGAGGGGGAGGCAG-3′,
(SEQ ID NO: 13)
5′-AGGGCGGTGTGGGAATAGGGAA-3′,
(SEQ ID NO: 15)
5′-CGGGGCGGGCCGGGGGCGGGGT-3′,
(SEQ ID NO: 23)
5′-GGGTAGGGGCGGGGCGGGGCGGGGGC-3′,
(SEQ ID NO: 20)
5′-GGAGGAGGAGGTCACGGAGGAGGAGGAGAAGGAGGAGGAGGA-3′,
(SEQ ID NO: 19)
5′-GGGAGGGAGAGGGGGCGGG-3′,
and,
(SEQ ID NO: 16)
5′-AGGGAGGGCGCTGGGAGGAGGG-3′.

5. The method of claim 2 wherein the G4 sequence is
(SEQ ID NO: 51)
5′-TGA₁₋₅GGGT₁₋₅GGG(GA)₁₋₅GGGT₁₋₅GGGGAA-3′,
or
(SEQ ID NO: 52)
5′-TGA₁₋₅GGGA₁₋₅GGGA₁₋₅GGGA₁₋₅GGGGAA-3′

6. The method of claim 2 wherein the G4 sequence is 5′-NNGGGTGGGGAGGGTGGGNN-3′ (SEQ ID NO: 3), where each N is independently selected in each instance from the group consisting of A, T, C, and G.

7. The method of claim 2 wherein the G4 sequence occurs in a human oncogene.

8. The method of claim 2 wherein the test compound is a protein, an oligopeptide, an oligonucleotide, or a small molecule.

9. The method of claim 8 wherein the test compound is a protein.

10. The method of claim 8 wherein the test compound is a small molecule.

11. A method for determining the binding preference of a test compound capable of providing a fluorescent signal (a fluorescent test compound) for one or more target G-quadruplex moieties, the method comprising the steps of;
a) incubating a device comprising a plurality of single-stranded nucleic acid molecules capable of forming one or more G-quadruplex moieties including the target G-quadruplex moieties with a solution comprising a G-quadruplex stabilizing cation selected from the group consisting of Na⁺ and K⁺,
b) contacting the fluorescent test compound with the device;
c) measuring a first fluorescent signal from the fluorescent test compound bound to the device;
d) incubating the device with a solution of solution of Li+;
e) contacting the fluorescent test compound with the device;
f) measuring a second fluorescent signal from the fluorescent test compound bound to the device;
g) using the first fluorescent signal and the second fluorescent signal to calculate the binding preference of the fluorescent test compound.

12. The method of claim 11 wherein the device is a microarray comprising a plurality of single-stranded DNA molecules (s-DNAs) attached to a solid substrate; where
each s-DNA is from 50 nt to 100 nt in length and
includes an independently selected linker sequence and an independently selected G-quadruplex-forming region (G4 sequence) where the G4 sequence has formula I
S1-T1-S2-T2-S3-T3-S4-T4-S5 (I) (SEQ ID NO: 54)
wherein T1 is G-Gx1, T2 is G-Gx2, T3 is G-Gx3, and T4 is G-Gx4;
S1 to S5 are independently selected sequences of from 0 to 5 nucleotides independently selected in each instance from the group consisting of A, T, C, and G; and
x1 to x4 are each independently selected from the group consisting of 2, 3, 4, and 5.

13. The method of claim 12 wherein the G-quadruplex stabilizing cation is K⁺.

14. The method of claim 12 wherein the G4 sequence is selected from the group consisting of
(SEQ ID NO: 42)
5′-TTATGGGGAGGGTGGGGAGGGTGGGGAAGGTGGGGAGGAG-3′,
(SEQ ID NO: 43)
5′-TTGGGGAGGGTGGGGAGGGTGGGGAAGGT-3′,
(SEQ ID NO: 10)
5′-TGGGGAGGGTGGGGAGGGTGGGGAAGG-3′,
(SEQ ID NO: 9)
5′-TTGGGGAGGGTGGGGAGGGTGGGGAA-3′,
(SEQ ID NO: 6)
5′-TGAGGGTGGGGAGGGTGGGGAA-3′,
(SEQ ID NO: 4)
5′-TGAGGGTGGGTAGGGTGGGTAA-3′,
(SEQ ID NO: 7)
5′-AGGGTGGGGAGGGTGGGG-3′,
(SEQ ID NO: 44)
5′-GCTGGGAGAAGGGGGGGCGGCGGGGCAGGGAGGGTGGACGC-3′,
(SEQ ID NO: 45)
5′-TTGGGAGAAGGGGGGGCGGCGGGGCA-3′,
(SEQ ID NO: 46)
5′-AAGGGAGGGCGGCGGGGCA-3′,
(SEQ ID NO: 47)
5′-AAGGGGGGGCGGCGGGGCAGGGAGGGT-3′,
(SEQ ID NO: 26)
5′-CGGCGGGGCAGGGAGGGTGGACG-3′,
(SEQ ID NO: 48)
5′-AGGGTTAGGGTTAGGGTTAGGG-3′,
(SEQ ID NO: 49)
5′-TTAGGGTTAGGGTTAGGGTTAGGGAAA-3′,
(SEQ ID NO: 50)
5′-TTAGGGTTAGGGTTAGGGTTAGGGTTA-3′,
(SEQ ID NO: 17)
5′-AGGGGCGGGCGCGGGAGGAAGGGGGCGGGA-3′,
(SEQ ID NO: 18)
5′-CGGGCGGGAGCGCGGCGGGCGGGCGGGC-3′,
(SEQ ID NO: 24)
5′-GGAGGCGGGGGGGGGGGGGCGGGGGCGGGGGCGGGGGAGGGGCG
CGGC-3′,
(SEQ ID NO: 12)
5′-AGGGCGGTGTGGGAAGAGGGAAGAGGGGGAGGCAG-3′,
(SEQ ID NO: 13)
5′-AGGGCGGTGTGGGAATAGGGAA-3′,
(SEQ ID NO: 15)
5′-CGGGGCGGGCCGGGGGCGGGGT-3′,
(SEQ ID NO: 23)
5′-GGGTAGGGGCGGGGCGGGGCGGGGGC-3′,
(SEQ ID NO: 20)
5′-GGAGGAGGAGGTCACGGAGGAGGAGGAGAAGGAGGAGGAGGA-3′,
(SEQ ID NO: 19)
5′-GGGAGGGAGAGGGGGCGGG-3′,
and,
(SEQ ID NO: 16)
5′-AGGGAGGGCGCTGGGAGGAGGG-3′.

15. The method of claim 12 wherein the G4 sequence is
(SEQ ID NO: 51)
5′-TGA₁₋₅GGGT₁₋₅GGG(GA)₁₋₅GGGT₁₋₅GGGGAA-3′,
or
(SEQ ID NO: 52)
5′-TGA₁₋₅GGGA₁₋₅GGGA₁₋₅GGGA₁₋₅GGGGAA-3′

16. The method of claim 12 wherein the G4 sequence is
(SEQ ID NO: 3)
5′-NNGGGTGGGGAGGGTGGGNN-3′
where each N is independently selected in each instance from the group consisting of A, T, C, and G.

17. The method of claim 12 wherein the G4 sequence occurs in a human oncogene.

18. The method of claim 12 wherein the test compound is a protein, an oligopeptide, an oligonucleotide, or a small molecule.

19. The method of claim 12 wherein the test compound is a protein.

20. The method of claim 12 wherein the test compound is a small molecule.
